SEQUENCE LISTING (328 sequences)

SEQ ID NO: 1 - protein, 5 aa, Artificial Sequence
  Human antibody library
LYWMH

SEQ ID NO: 2 - protein, 7 aa, Artificial Sequence
  Human antibody library
GFTFSLY

SEQ ID NO: 3 - protein, 17 aa, Artificial Sequence
  Human antibody library
SIYSSGVMTFYADSVKG

SEQ ID NO: 4 - protein, 6 aa, Artificial Sequence
  Human antibody library
YSSGVM

SEQ ID NO: 5 - protein, 9 aa, Artificial Sequence
  Human antibody library
GSRVDAFDI

SEQ ID NO: 6 - protein, 118 aa, Artificial Sequence
  Human antibody library
EVQLLESGGGLVQPGGSLRLSCAASGFTFSLYWMHWVRQAPGKGLEWVSSIYSSGVMTFYADSVKGRFTISRDNSKNTLYLQMNSLRAEDTAVYYCARGSRVDAFDIWGQGTMVTVSS

SEQ ID NO: 7 - protein, 12 aa, Artificial Sequence
  Human antibody library
RASQSVSSSYLA

SEQ ID NO: 8 - protein, 7 aa, Artificial Sequence
  Human antibody library
GASARAI

SEQ ID NO: 9 - protein, 9 aa, Artificial Sequence
  Human antibody library
QQSYSIPIT

SEQ ID NO: 10 - protein, 108 aa, Artificial Sequence
  Human antibody library
DIQMTQSPGTLSLSPGERATLSCRASQSVSSSYLAWYQQKPGQAPRLLIYGASARAIGVPDRFRGSGSGTDFTLTISSLQPEDFATYYCQQSYSIPITFGPGTKVDIK

SEQ ID NO: 11 - protein, 5 aa, Artificial Sequence
  Human antibody library
PYPMW

SEQ ID NO: 12 - protein, 7 aa, Artificial Sequence
  Human antibody library
GFTFSPY

SEQ ID NO: 13 - protein, 17 aa, Artificial Sequence
  Human antibody library
SISPSGGFTFYADSVKG

SEQ ID NO: 14 - protein, 6 aa, Artificial Sequence
  Human antibody library
SPSGGF

SEQ ID NO: 15 - protein, 13 aa, Artificial Sequence
  Human antibody library
DRVSWNDYTGMDV

SEQ ID NO: 16 - protein, 122 aa, Artificial Sequence
  Human antibody library
EVQLLESGGGLVQPGGSLRLSCAASGFTFSPYPMWWVRQAPGKGLEWVSSISPSGGFTFYADSVKGRFTISRDNSKNTLYLQMNSLRAEDTAVYYCARDRVSWNDYTGMDVWGQGTTVTVSS

SEQ ID NO: 17 - protein, 11 aa, Artificial Sequence
  Human antibody library
RASESIGKFLN

SEQ ID NO: 18 - protein, 7 aa, Artificial Sequence
  Human antibody library
SASSLQS

SEQ ID NO: 19 - protein, 9 aa, Artificial Sequence
  Human antibody library
QQSFDMPIT

SEQ ID NO: 20 - protein, 107 aa, Artificial Sequence
  Human antibody library
DIQMTQSPSSLSASVGDRVTITCRASESIGKFLNWYQQKPGKAPTLLIHSASSLQSGVPSRFSGSRSGTDFTLTISSLQPEDFTTYFCQQSFDMPITFGQGTQLEIK

SEQ ID NO: 21 - protein, 5 aa, Artificial Sequence
  Human antibody library
AYIMG

SEQ ID NO: 22 - protein, 7 aa, Artificial Sequence
  Human antibody library
GFTFSAY

SEQ ID NO: 23 - protein, 17 aa, Artificial Sequence
  Human antibody library
YISSSGGWTAYADSVKG

SEQ ID NO: 24 - protein, 6 aa, Artificial Sequence
  Human antibody library
SSSGGW

SEQ ID NO: 25 - protein, 10 aa, Artificial Sequence
  Human antibody library
DQDGYNDFDY

SEQ ID NO: 26 - protein, 119 aa, Artificial Sequence
  Human antibody library
EVQLLESGGGLVQPGGSLRLSCAASGFTFSAYIMGWVRQAPGKGLEWVSYISSSGGWTAYADSVKGRFTISRDNSKNTLYLQMNSLRAEDTAVYYCATDQDGYNDFDYWGQGTLVTVSS

SEQ ID NO: 27 - protein, 11 aa, Artificial Sequence
  Human antibody library
RASQSISSYLN

SEQ ID NO: 28 - protein, 7 aa, Artificial Sequence
  Human antibody library
AASSLQS

SEQ ID NO: 29 - protein, 9 aa, Artificial Sequence
  Human antibody library
QQSYSTPRT

SEQ ID NO: 30 - protein, 107 aa, Artificial Sequence
  Human antibody library
DIQMTQSPSSLSASVGDRVTITCRASQSISSYLNWYQQKPGKAPNLLIYAASSLQSGVPSRFSGSGSGTDFTLTISSLQPEDFATYYCQQSYSTPRTFGHGTKVEIK

SEQ ID NO: 31 - protein, 5 aa, Artificial Sequence
  Human antibody library
MYWMA

SEQ ID NO: 32 - protein, 7 aa, Artificial Sequence
  Human antibody library
GFTFSMY

SEQ ID NO: 33 - protein, 17 aa, Artificial Sequence
  Human antibody library
YIVPSGGITLYADSVKG

SEQ ID NO: 34 - protein, 6 aa, Artificial Sequence
  Human antibody library
VPSGGI

SEQ ID NO: 35 - protein, 9 aa, Artificial Sequence
  Human antibody library
QVRGGAFDI

SEQ ID NO: 36 - protein, 118 aa, Artificial Sequence
  Human antibody library
EVQLLESGGGLVQPGGSLRLSCAASGFTFSMYWMAWVRQAPGKGLEWVSYIVPSGGITLYADSVKGRFTISRDNSKNTLYLQMNSLRAEDTAMYYCARQVRGGAFDIWGQGTMVTVSS

SEQ ID NO: 37 - protein, 11 aa, Artificial Sequence
  Human antibody library
QASHDISNYLN

SEQ ID NO: 38 - protein, 7 aa, Artificial Sequence
  Human antibody library
DASNLET

SEQ ID NO: 39 - protein, 9 aa, Artificial Sequence
  Human antibody library
QHYDNLPPS

SEQ ID NO: 40 - protein, 107 aa, Artificial Sequence
  Human antibody library
DIQMTQSPSSLSASVGDRVTITCQASHDISNYLNWYQQKPGKAPKLLIYDASNLETGVPSRFSGSGSGTDFTFTISSLQPEDIATYYCQHYDNLPPSFGQGTKLEIK

SEQ ID NO: 41 - protein, 5 aa, Artificial Sequence
  from human Fab library
HYPMV

SEQ ID NO: 42 - protein, 7 aa, Artificial Sequence
  from human Fab library
GFTFSHY

SEQ ID NO: 43 - protein, 17 aa, Artificial Sequence
  from human Fab library
WIGSSGGFTMYADSVKG

SEQ ID NO: 44 - protein, 6 aa, Artificial Sequence
  from human Fab library
GSSGGF

SEQ ID NO: 45 - protein, 11 aa, Artificial Sequence
  Human antibody library
DSFEDHYYMDV

SEQ ID NO: 46 - protein, 120 aa, Artificial Sequence
  Human antibody library
EVQLLESGGGLVQPGGSLRLSCAASGFTFSHYPMVWVRQAPGKGLEWVSWIGSSGGFTMYADSVKGRFTISRDNSKNTLYLQMNSLRAEDTAVYYCTRDSFEDHYYMDVWGKGTTVTVSS

SEQ ID NO: 47 - protein, 16 aa, Artificial Sequence
  Human antibody library
RSSQSLLHSNGYNYLD

SEQ ID NO: 48 - protein, 7 aa, Artificial Sequence
  Human antibody library
LGSNRAS

SEQ ID NO: 49 - protein, 9 aa, Artificial Sequence
  Human antibody library
MQALQTPLT

SEQ ID NO: 50 - protein, 112 aa, Artificial Sequence
  Human antibody library
DIQMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 51 - protein, 5 aa, Artificial Sequence
  Human antibody library
EYVMI

SEQ ID NO: 52 - protein, 7 aa, Artificial Sequence
  Human antibody library
GFTFSEY

SEQ ID NO: 53 - protein, 17 aa, Artificial Sequence
  Human antibody library
SISSSGGFTWYADSVKG

SEQ ID NO: 54 - protein, 6 aa, Artificial Sequence
  Human antibody library
SSSGGF

SEQ ID NO: 55 - protein, 12 aa, Artificial Sequence
  Human antibody library
ARAPVGADAFDI

SEQ ID NO: 56 - protein, 121 aa, Artificial Sequence
  Human antibody library
EVQLLESGGGLVQPGGSLRLSCAASGFTFSEYVMIWVRQAPGKGLEWVSSISSSGGFTWYADSVKGRFTISRDNSKNTLYLQMNSLRAEDTAVYYCARARAPVGADAFDIWGQGTMVTVSS

SEQ ID NO: 57 - protein, 11 aa, Artificial Sequence
  Human antibody library
RASQRISSYLN

SEQ ID NO: 58 - protein, 7 aa, Artificial Sequence
  Human antibody library
NAASLWN

SEQ ID NO: 59 - protein, 9 aa, Artificial Sequence
  Human antibody library
QQSHSSPYT

SEQ ID NO: 60 - protein, 107 aa, Artificial Sequence
  Human antibody library
DIQMTQSPSSLSASVGDRVTITCRASQRISSYLNWYQQKPGQAPKLLIDNAASLWNGVPSRFSGSGSGTDFTLTITTLMPEDFATYFCQQSHSSPYTFGQGTKLEIR

SEQ ID NO: 61 - protein, 5 aa, Artificial Sequence
  from human Fab library
SYEMH

SEQ ID NO: 62 - protein, 7 aa, Artificial Sequence
  from human Fab library
GFTFSSY

SEQ ID NO: 63 - protein, 17 aa, Artificial Sequence
  from human Fab library
GIWPSGGVTLYADSVKG

SEQ ID NO: 64 - protein, 6 aa, Artificial Sequence
  from human Fab library
WPSGGV

SEQ ID NO: 65 - protein, 8 aa, Artificial Sequence
  Human antibody library
FRTQPFDI

SEQ ID NO: 66 - protein, 117 aa, Artificial Sequence
  Human antibody library
EVQLLESGGGLVQPGGSLRLSCAASGFTFSSYEMHWVRQAPGKGLEWVSGIWPSGGVTLYADSVKGRFTISRDNSKNTLYLQMNSLRAEDTAVYYCARFRTQPFDIWGQGTMVTVSS

SEQ ID NO: 67 - protein, 11 aa, Artificial Sequence
  Human antibody library
RASQGISNYLA

SEQ ID NO: 68 - protein, 7 aa, Artificial Sequence
  Human antibody library
AASTLQS

SEQ ID NO: 69 - protein, 8 aa, Artificial Sequence
  Human antibody library
HNYNSALT

SEQ ID NO: 70 - protein, 106 aa, Artificial Sequence
  Human antibody library
DIQMTQSPDSLSASVGDRVTITCRASQGISNYLAWYQQKPGKVPKLLIYAASTLQSGVPSRFSGSGSGTDFTLTISSLQPEDVATYFCHNYNSALTFGGGTKVEIK

SEQ ID NO: 71 - protein, 5 aa, Artificial Sequence
  Human antibody library
RYGMG

SEQ ID NO: 72 - protein, 7 aa, Artificial Sequence
  Human antibody library
GFTFSRY

SEQ ID NO: 73 - protein, 17 aa, Artificial Sequence
  Human antibody library
SISSSGGQTSYADSVKG

SEQ ID NO: 74 - protein, 6 aa, Artificial Sequence
  Human antibody library
SSSGGQ

SEQ ID NO: 75 - protein, 9 aa, Artificial Sequence
  Human antibody library
GGLWFGLDP

SEQ ID NO: 76 - protein, 118 aa, Artificial Sequence
  Human antibody library
EVQLLESGGGLVQPGGSLRLSCAASGFTFSRYGMGWVRQAPGKGLEWVSSISSSGGQTSYADSVKGRFTISRDNSKNTLYLQMNSLRAEDTAVYYCTRGGLWFGLDPWGQGTLVTVSS

SEQ ID NO: 77 - protein, 11 aa, Artificial Sequence
  Human antibody library
RASQGISTWLA

SEQ ID NO: 78 - protein, 7 aa, Artificial Sequence
  Human antibody library
AASSLQS

SEQ ID NO: 79 - protein, 9 aa, Artificial Sequence
  Human antibody library
QQANSFPST

SEQ ID NO: 80 - protein, 107 aa, Artificial Sequence
  Human antibody library
DIQMTQSPSSVSASVGDRVTITCRASQGISTWLAWYQQKPGKAPKLLIYAASSLQSGVPSRFSGSGSGTDFTLSISSLQPEDFATYYCQQANSFPSTFGGGTKVEIK

SEQ ID NO: 81 - protein, 5 aa, Artificial Sequence
  Human antibody library
KYVMH

SEQ ID NO: 82 - protein, 7 aa, Artificial Sequence
  Human antibody library
GFTFSKY

SEQ ID NO: 83 - protein, 17 aa, Artificial Sequence
  Human antibody library
YISSSGGFTVYADSVKG

SEQ ID NO: 84 - protein, 6 aa, Artificial Sequence
  from human Fab library
SSSGGF

SEQ ID NO: 85 - protein, 10 aa, Artificial Sequence
  Human antibody library
VFDSGDAFDI

SEQ ID NO: 86 - protein, 119 aa, Artificial Sequence
  Human antibody library
EVQLLESGGGLVQPGGSLRLSCAASGFTFSKYVMHWVRQAPGKGLEWVSYISSSGGFTVYADSVKGRFTISRDNSKNTLYLQMNSLRAEDTAVYYCARVFDSGDAFDIWGQGTMVTVSS

SEQ ID NO: 87 - protein, 17 aa, Artificial Sequence
  Human antibody library
KSSQSVLYSSNNNNYLA

SEQ ID NO: 88 - protein, 7 aa, Artificial Sequence
  Human antibody library
WASTRES

SEQ ID NO: 89 - protein, 9 aa, Artificial Sequence
  Human antibody library
QQYYSTPPT

SEQ ID NO: 90 - protein, 113 aa, Artificial Sequence
  from human Fab library
DIQMTQSPDSLAVSLGERATINCKSSQSVLYSSNNNNYLAWYQQKPGQPPKLLIYWASTRESGVPDRFSGSGSGTDFTLTISSLQAEDVAVYYCQQYYSTPPTFGQGTKVEIK

SEQ ID NO: 91 - protein, 5 aa, Artificial Sequence
  from human Fab library
WYPMH

SEQ ID NO: 92 - protein, 7 aa, Artificial Sequence
  from human Fab library
GFTFSWY

SEQ ID NO: 93 - protein, 17 aa, Artificial Sequence
  from human Fab library
SISSSGGFTMYADSVKG

SEQ ID NO: 94 - protein, 6 aa, Artificial Sequence
  from human Fab library
SSSGGF

SEQ ID NO: 95 - protein, 11 aa, Artificial Sequence
  from human Fab library
EGGYSYGPGDY

SEQ ID NO: 96 - protein, 120 aa, Artificial Sequence
  Human antibody library
EVQLLESGGGLVQPGGSLRLSCAASGFTFSWYPMHWVRQAPGKGLEWVSSISSSGGFTMYADSVKGRFTISRDNSKNTLYLQMNSLRAEDTAMYYCAKEGGYSYGPGDYWGQGTLVTVSS

SEQ ID NO: 97 - protein, 11 aa, Artificial Sequence
  Human antibody library
RASQGISSWLA

SEQ ID NO: 98 - protein, 7 aa, Artificial Sequence
  Human antibody library
AASSLQS

SEQ ID NO: 99 - protein, 9 aa, Artificial Sequence
  Human antibody library
QQANSFPYT

SEQ ID NO: 100 - protein, 107 aa, Artificial Sequence
  from human Fab library
DIQMTQSPSSVSASVGDRVTITCRASQGISSWLAWYQQKPGKAPKLLIYAASSLQSGVPSRFSGSGSGTDFTLTISSLQPEDFATYYCQQANSFPYTFGQGTKLEIK

SEQ ID NO: 101 - protein, 5 aa, Artificial Sequence
  from human Fab library
WYPMT

SEQ ID NO: 102 - protein, 7 aa, Artificial Sequence
  from human Fab library
GFTFSWY

SEQ ID NO: 103 - protein, 17 aa, Artificial Sequence
  from human Fab library
SIGSSGGFTLYADSVKG

SEQ ID NO: 104 - protein, 7 aa, Artificial Sequence
  Human antibody library
IGSSGGF

SEQ ID NO: 105 - protein, 12 aa, Artificial Sequence
  Human antibody library
DVWGIAAPYFDY

SEQ ID NO: 106 - protein, 121 aa, Artificial Sequence
  Human antibody library
EVQLLESGGGLVQPGGSLRLSCAASGFTFSWYPMTWVRQAPGKGLEWVSSIGSSGGFTLYADSVKGRFTISRDNSKNTLYLQMNSLRAEDTAVYYCTRDVWGIAAPYFDYWGQGTLVTVSS

SEQ ID NO: 107 - protein, 11 aa, Artificial Sequence
  Human antibody library
RASQGIGSWLG

SEQ ID NO: 108 - protein, 7 aa, Artificial Sequence
  Human antibody library
AASNLQS

SEQ ID NO: 109 - protein, 9 aa, Artificial Sequence
  Human antibody library
QQVNNFPRA

SEQ ID NO: 110 - protein, 107 aa, Artificial Sequence
  Human antibody library
DIQMTQSPSSVSASVGDRVTISCRASQGIGSWLGWYQQKPGKAPKLLIYAASNLQSGVPSRFSGSGSGTDFTLTISNLQPEDFATYYCQQVNNFPRAFGGGTKVEIK

SEQ ID NO: 111 - protein, 5 aa, Artificial Sequence
  Human antibody library
WYIMA

SEQ ID NO: 112 - protein, 7 aa, Artificial Sequence
  from human Fab library
GFTFSWY

SEQ ID NO: 113 - protein, 17 aa, Artificial Sequence
  Human antibody library
YISSSGGFTAYADSVKG

SEQ ID NO: 114 - protein, 6 aa, Artificial Sequence
  Human antibody library
SSSGGF

SEQ ID NO: 115 - protein, 9 aa, Artificial Sequence
  Human antibody library
IGGTDVFDI

SEQ ID NO: 116 - protein, 118 aa, Artificial Sequence
  Human antibody library
EVQLLESGGGLVQPGGSLRLSCAASGFTFSWYIMAWVRQAPGKGLEWVSYISSSGGFTAYADSVKGRFTISRDNSKNTLYLQMNSLRAEDTAVYYCARIGGTDVFDIWGQGTLVTVSS

SEQ ID NO: 117 - protein, 11 aa, Artificial Sequence
  Human antibody library
RASQSISSWLA

SEQ ID NO: 118 - protein, 7 aa, Artificial Sequence
  Human antibody library
KASSLES

SEQ ID NO: 119 - protein, 9 aa, Artificial Sequence
  Human antibody library
QQSYSSPWT

SEQ ID NO: 120 - protein, 107 aa, Artificial Sequence
  Human antibody library
DIQMTQSPSTLSASVGDRVTITCRASQSISSWLAWYQQKPGKAPKLLIYKASSLESGVPSRFSGSGSGTDFTLTITSLLPEDFATYYCQQSYSSPWTFGQGTKVEVK

SEQ ID NO: 121 - protein, 5 aa, Artificial Sequence
  Human antibody library
WYLMK

SEQ ID NO: 122 - protein, 7 aa, Artificial Sequence
  Human antibody library
GFTFSWY

SEQ ID NO: 123 - protein, 17 aa, Artificial Sequence
  Human antibody library
YIGSSGGFTAYADSVKG

SEQ ID NO: 124 - protein, 6 aa, Artificial Sequence
  Human antibody library
GSSGGF

SEQ ID NO: 125 - protein, 9 aa, Artificial Sequence
  Human antibody library
EDDFGAMDV

SEQ ID NO: 126 - protein, 118 aa, Artificial Sequence
  Human antibody library
EVQLLESGGGLVQPGGSLRLSCAASGFTFSWYLMKWVRQAPGKGLEWVSYIGSSGGFTAYADSVKGRFTISRDNSKNTLYLQMNSLRAEDTAMYYCAREDDFGAMDVWGQGTTVTVSS

SEQ ID NO: 127 - protein, 11 aa, Artificial Sequence
  Human antibody library
RASQTVSKYFN

SEQ ID NO: 128 - protein, 7 aa, Artificial Sequence
  Human antibody library
ATSTLQS

SEQ ID NO: 129 - protein, 9 aa, Artificial Sequence
  Human antibody library
QQSYTTPWT

SEQ ID NO: 130 - protein, 107 aa, Artificial Sequence
  Human antibody library
DIQMTQSPSSLSASVGDRVTITCRASQTVSKYFNWFQQKPGEAPKLLIYATSTLQSGVPSRFSGSGYGTEFTLTISSLQPEDFATYYCQQSYTTPWTFGQGTKVEIK

SEQ ID NO: 131 - protein, 5 aa, Artificial Sequence
  Human antibody library
SYQMG

SEQ ID NO: 132 - protein, 7 aa, Artificial Sequence
  Human antibody library
GFTFSSY

SEQ ID NO: 133 - protein, 17 aa, Artificial Sequence
  Human antibody library
WIVPSGGFTHYADSVKG

SEQ ID NO: 134 - protein, 6 aa, Artificial Sequence
  Human antibody library
VPSGGF

SEQ ID NO: 135 - protein, 16 aa, Artificial Sequence
  Human antibody library
DAGYRSGYYYYYGMDV

SEQ ID NO: 136 - protein, 125 aa, Artificial Sequence
  Human antibody library
EVQLLESGGGLVQPGGSLRLSCAASGFTFSSYQMGWVRQAPGKGLEWVSWIVPSGGFTHYADSVKGRFTISRDNSKNTLYLQMNSLRAEDTAVYYCARDAGYRSGYYYYYGMDVWGQGTTVTVSS

SEQ ID NO: 137 - protein, 11 aa, Artificial Sequence
  Human antibody library
RASQSIASYLN

SEQ ID NO: 138 - protein, 7 aa, Artificial Sequence
  Human antibody library
GASSLQS

SEQ ID NO: 139 - protein, 9 aa, Artificial Sequence
  Human antibody library
QQSYGISYT

SEQ ID NO: 140 - protein, 107 aa, Artificial Sequence
  Human antibody library
DIQMTQSPSSLSASVGDRVTITCRASQSIASYLNWYQQKPGKAPKLLIYGASSLQSGVPSRFSGSGSGTDFTLTIRSLQPEDIATYYCQQSYGISYTFGQGTKLEIK

SEQ ID NO: 141 - protein, 5 aa, Artificial Sequence
  Human antibody library
HYPMS

SEQ ID NO: 142 - protein, 7 aa, Artificial Sequence
  Human antibody library
GFTFSHY

SEQ ID NO: 143 - protein, 17 aa, Artificial Sequence
  Human antibody library
RIWSSGGNTYYADSVKG

SEQ ID NO: 144 - protein, 7 aa, Artificial Sequence
  Human antibody library
IWSSGGN

SEQ ID NO: 145 - protein, 12 aa, Artificial Sequence
  from human antibody library
GGYFDWLYPHDY

SEQ ID NO: 146 - protein, 121 aa, Artificial Sequence
  from human antibody library
EVQLLESGGGLVQPGGSLRLSCAASGFTFSHYPMSWVRQAPGKGLEWVSRIWSSGGNTYYADSVKGRFTISRDNSKNTLYLQMNSLRAEDTAVYYCARGGYFDWLYPHDYWGQGTLVTVSS

SEQ ID NO: 147 - protein, 11 aa, Artificial Sequence
  from human antibody library
RASQGISSWLS

SEQ ID NO: 148 - protein, 7 aa, Artificial Sequence
  from human antibody library
AASSLQS

SEQ ID NO: 149 - protein, 9 aa, Artificial Sequence
  Human antibody library
QQGNSFPPT

SEQ ID NO: 150 - protein, 107 aa, Artificial Sequence
  Human antibody library
DIQMTQSPSSVSASVGDRVTITCRASQGISSWLSWYQQKPGAAPKLLIYAASSLQSGVPARFSGSGSGREFTLTISNLQPEDFATYYCQQGNSFPPTFGQGTKVDIK

SEQ ID NO: 151 - protein, 5 aa, Artificial Sequence
  Human antibody library
RYEML

SEQ ID NO: 152 - protein, 7 aa, Artificial Sequence
  Human antibody library
GFTFSRY

SEQ ID NO: 153 - protein, 17 aa, Artificial Sequence
  Human antibody library
SIYSSGGWTWYADSVKG

SEQ ID NO: 154 - protein, 6 aa, Artificial Sequence
  Human antibody library
YSSGGW

SEQ ID NO: 155 - protein, 10 aa, Artificial Sequence
  Human antibody library
HSVTGVAFDY

SEQ ID NO: 156 - protein, 119 aa, Artificial Sequence
  Human antibody library
EVQLLESGGGLVQPGGSLRLSCAASGFTFSRYEMLWVRQAPGKGLEWVSSIYSSGGWTWYADSVKGRFTISRDNSKNTLYLQMNSLRAEDTAMYYCARHSVTGVAFDYWGQGTLVTVSS

SEQ ID NO: 157 - protein, 11 aa, Artificial Sequence
  Human antibody library
RASQNIDTYLS

SEQ ID NO: 158 - protein, 7 aa, Artificial Sequence
  Human antibody library
AASSLQS

SEQ ID NO: 159 - protein, 8 aa, Artificial Sequence
  Human antibody library
QQSYYTPT

SEQ ID NO: 160 - protein, 106 aa, Artificial Sequence
  Human antibody library
DIQMTQSPSSLSASVGDRVTITCRASQNIDTYLSWYQQKPGKAPKLLIYAASSLQSGVPSRFSGSRSGTDFTLTISSLQPEDFATYYCQQSYYTPTFGGGTKVEIK

SEQ ID NO: 161 - protein, 5 aa, Artificial Sequence
  from human antibody library
PYDMG

SEQ ID NO: 162 - protein, 7 aa, Artificial Sequence
  Human antibody library
GFTFSPY

SEQ ID NO: 163 - protein, 17 aa, Artificial Sequence
  Human antibody library
SIYSSGGWTKYADSVKG

SEQ ID NO: 164 - protein, 6 aa, Artificial Sequence
  Human antibody library
YSSGGW

SEQ ID NO: 165 - protein, 10 aa, Artificial Sequence
  Human antibody library
DNWNDGAFDV

SEQ ID NO: 166 - protein, 119 aa, Artificial Sequence
  Human antibody library
EVQLLESGGGLVQPGGSLRLSCAASGFTFSPYDMGWVRQAPGKGLEWVSSIYSSGGWTKYADSVKGRFTISRDNSKNTLYLQMNSLRAEDTAVYYCARDNWNDGAFDVWGQGTMVTVSS

SEQ ID NO: 167 - protein, 11 aa, Artificial Sequence
  Human antibody library
RASQSISSWLA

SEQ ID NO: 168 - protein, 7 aa, Artificial Sequence
  Human antibody library
AASRLQT

SEQ ID NO: 169 - protein, 9 aa, Artificial Sequence
  Human antibody library
QQAKTFPLT

SEQ ID NO: 170 - protein, 107 aa, Artificial Sequence
  Human antibody library
DIQMTQSPSTLSASVGDRVTITCRASQSISSWLAWYQQKPGKAPKLLIFAASRLQTGVPSRFSGSGSGTDFTLTISSLQPEDSATYYCQQAKTFPLTFGGGTRVEIK

SEQ ID NO: 171 - protein, 5 aa, Artificial Sequence
  Human antibody library
FYDMS

SEQ ID NO: 172 - protein, 7 aa, Artificial Sequence
  Human antibody library
GFTFSFY

SEQ ID NO: 173 - protein, 17 aa, Artificial Sequence
  Human antibody library
SIVPSGGWTFYADSVKG

SEQ ID NO: 174 - protein, 6 aa, Artificial Sequence
  Human antibody library
VPSGGW

SEQ ID NO: 175 - protein, 10 aa, Artificial Sequence
  Human antibody library
DSWNDGASDI

SEQ ID NO: 176 - protein, 119 aa, Artificial Sequence
  Human antibody library
EVQLLESGGGLVQPGGSLRLSCAASGFTFSFYDMSWVRQAPGKGLEWVSSIVPSGGWTFYADSVKGRFTISRDNSKNTLYLQMNSLRAEDTAVYYCARDSWNDGASDIWGQGTMVTVSS

SEQ ID NO: 177 - protein, 11 aa, Artificial Sequence
  from human antibody library
RASQGISSWLA

SEQ ID NO: 178 - protein, 7 aa, Artificial Sequence
  from human antibody library
AASSLQS

SEQ ID NO: 179 - protein, 9 aa, Artificial Sequence
  from human antibody library
QQANSFPFT

SEQ ID NO: 180 - protein, 107 aa, Artificial Sequence
  from human antibody library
DIQMTQSPSSVSASVGDRVTITCRASQGISSWLAWYQQKPGKAPKLLIYAASSLQSGVPSRFSGSGSGTDFTLTINSLQPEDFATYYCQQANSFPFTFGPGTKVDFK

SEQ ID NO: 181 - protein, 5 aa, Artificial Sequence
  Human antibody library
PYGMR

SEQ ID NO: 182 - protein, 7 aa, Artificial Sequence
  Human antibody library
GFTFSPY

SEQ ID NO: 183 - protein, 17 aa, Artificial Sequence
  Human antibody library
SISPSGGNTDYADSVKG

SEQ ID NO: 184 - protein, 6 aa, Artificial Sequence
  Human antibody library
SPSGGN

SEQ ID NO: 185 - protein, 12 aa, Artificial Sequence
  Human antibody library
RASHSVSSDYLA

SEQ ID NO: 186 - protein, 16 aa, Artificial Sequence
  Human antibody library
IRYCGSAYCYTDAFDI

SEQ ID NO: 187 - protein, 11 aa, Artificial Sequence
  Human antibody library
SGEKLGDRYVS

SEQ ID NO: 188 - protein, 7 aa, Artificial Sequence
  from human Fab library
HDKKRPP

SEQ ID NO: 189 - protein, 9 aa, Artificial Sequence
  from human antibody library
QAWDSPTEV

SEQ ID NO: 190 - protein, 109 aa, Artificial Sequence
  from human antibody library
QSALTQAPSVSVSPGQTTTITCSGEKLGDRYVSWYQQKPDQSPILVLYHDKKRPPGIPERFSGSNSGDTATLTITGTHTMDEADYYCQAWDSPTEVFGPGTKLTVLSQP

SEQ ID NO: 191 - protein, 5 aa, Artificial Sequence
  from human antibody library
MYDMA

SEQ ID NO: 192 - protein, 7 aa, Artificial Sequence
  from human antibody library
GFTFSMY

SEQ ID NO: 193 - protein, 17 aa, Artificial Sequence
  Human antibody library
GIWPSGGPTMYADSVKG

SEQ ID NO: 194 - protein, 6 aa, Artificial Sequence
  Human antibody library
WPSGGP

SEQ ID NO: 195 - protein, 10 aa, Artificial Sequence
  Human antibody library
GYSYGDALDY

SEQ ID NO: 196 - protein, 119 aa, Artificial Sequence
  Human antibody library
EVQLLESGGGLVQPGGSLRLSCAASGFTFSMYDMAWVRQAPGKGLEWVSGIWPSGGPTMYADSVKGRFTISRDNSKNTLYLQMNSLRAEDTAVYYCAGGYSYGDALDYWGQGTLVTVSS

SEQ ID NO: 197 - protein, 13 aa, Artificial Sequence
  Human antibody library
SGTSSNIGRNYVS

SEQ ID NO: 198 - protein, 7 aa, Artificial Sequence
  Human antibody library
DDRNRPS

SEQ ID NO: 199 - protein, 11 aa, Artificial Sequence
  Human antibody library
GTWDTSLSVVV

SEQ ID NO: 200 - protein, 113 aa, Artificial Sequence
  Human antibody library
QSALTQPPSASGSPGQSVTISCSGTSSNIGRNYVSWYRHLPGTAPKLLIYDDRNRPSGIVDRFSGSKSGTSATLAITGLQTGDEADYYCGTWDTSLSVVVFGGGTKVTVLSQP

SEQ ID NO: 201 - protein, 5 aa, Artificial Sequence
  Human antibody library
EYRMI

SEQ ID NO: 202 - protein, 7 aa, Artificial Sequence
  Human antibody library
GFTFSEY

SEQ ID NO: 203 - protein, 17 aa, Artificial Sequence
  Human antibody library
GIYPSGGWTNYADSVKG

SEQ ID NO: 204 - protein, 6 aa, Artificial Sequence
  Human antibody library
YPSGGW

SEQ ID NO: 205 - protein, 9 aa, Artificial Sequence
  Human antibody library
IGGANAFDI

SEQ ID NO: 206 - protein, 118 aa, Artificial Sequence
  Human antibody library
EVQLLESGGGLVQPGGSLRLSCAASGFTFSEYRMIWVRQAPGKGLEWVSGIYPSGGWTNYADSVKGRFTISRDNSKNTLYLQMNSLRAEDTAVYYCARIGGANAFDIWGQGTMVTVSS

SEQ ID NO: 207 - protein, 14 aa, Artificial Sequence
  Human antibody library
TGTSSDIGGYNYVS

SEQ ID NO: 208 - protein, 7 aa, Artificial Sequence
  Human antibody library
EVSNRPS

SEQ ID NO: 209 - protein, 11 aa, Artificial Sequence
  Human antibody library
SSYTSGSTRYV

SEQ ID NO: 210 - protein, 114 aa, Artificial Sequence
  Human antibody library
QSALTQPASVSGSPGQSITISCTGTSSDIGGYNYVSWYQQHPGKAPKLMIYEVSNRPSGVSNRFSGSKSGNTASLTISGLQAEDEADYYCSSYTSGSTRYVFGPGTKVTVLGQP

SEQ ID NO: 211 - protein, 5 aa, Artificial Sequence
  Human antibody library
KYRMA

SEQ ID NO: 212 - protein, 7 aa, Artificial Sequence
  Human antibody library
GFTFSKY

SEQ ID NO: 213 - protein, 17 aa, Artificial Sequence
  Human antibody library
YIYPSGGFTFYADSVKG

SEQ ID NO: 214 - protein, 6 aa, Artificial Sequence
  Human antibody library
YPSGGF

SEQ ID NO: 215 - protein, 11 aa, Artificial Sequence
  Human antibody library
DIGQWLFVFDY

SEQ ID NO: 216 - protein, 120 aa, Artificial Sequence
  Human antibody library
EVQLLESGGGLVQPGGSLRLSCAASGFTFSKYRMAWVRQAPGKGLEWVSYIYPSGGFTFYADSVKGRFTISRDNSKNTLYLQMNSLRAEDTAVYYCAKDIGQWLFVFDYWGQGTLVTVSS

SEQ ID NO: 217 - protein, 14 aa, Artificial Sequence
  Human antibody library
TGTSSDVGGYNYVS

SEQ ID NO: 218 - protein, 7 aa, Artificial Sequence
  Human antibody library
DVSNRPS

SEQ ID NO: 219 - protein, 10 aa, Artificial Sequence
  Human antibody library
SSYTNTITVV

SEQ ID NO: 220 - protein, 113 aa, Artificial Sequence
  Human antibody library
QSELTQPASVSGSPGQSITISCTGTSSDVGGYNYVSWYQQHPGKAPKLMIYDVSNRPSGVSNRFSGSKSGNTASLTISGLQAEDEADYYCSSYTNTITVVFGGGTKLTVLGQP

SEQ ID NO: 221 - protein, 5 aa, Artificial Sequence
  Human antibody library
KYDMY

SEQ ID NO: 222 - protein, 7 aa, Artificial Sequence
  Human antibody library
GFTFSKY

SEQ ID NO: 223 - protein, 17 aa, Artificial Sequence
  Human antibody library
GIWPSGGLTMYADSVKG

SEQ ID NO: 224 - protein, 6 aa, Artificial Sequence
  Human antibody library
WPSGGL

SEQ ID NO: 225 - protein, 12 aa, Artificial Sequence
  Human antibody library
DGVVGGSYAFDI

SEQ ID NO: 226 - protein, 121 aa, Artificial Sequence
  Human antibody library
EVQLLESGGGLVQPGGSLRLSCAASGFTFSKYDMYWVRQAPGKGLEWVSGIWPSGGLTMYADSVKGRFTISRDNSKNTLYLQMNSLRAEDTAMYYCARDGVVGGSYAFDIWGQGTMVTVSS

SEQ ID NO: 227 - protein, 13 aa, Artificial Sequence
  Human antibody library
SGASSNLGRNTVN

SEQ ID NO: 228 - protein, 7 aa, Artificial Sequence
  Human antibody library
TNDHRPS

SEQ ID NO: 229 - protein, 11 aa, Artificial Sequence
  Human antibody library
AAWDHSLNGYV

SEQ ID NO: 230 - protein, 113 aa, Artificial Sequence
  Human antibody library
QSALTQPPSASGTPGQRVTISCSGASSNLGRNTVNWYQQVPGSAPKLLIYTNDHRPSGVPDRFSGSRSGTSASLAISGLQSEDEADYYCAAWDHSLNGYVFGPGTKVTVLSQP

SEQ ID NO: 231 - protein, 5 aa, Artificial Sequence
  Human antibody library
IYSMN

SEQ ID NO: 232 - protein, 7 aa, Artificial Sequence
  Human antibody library
GFTFSIY

SEQ ID NO: 233 - protein, 17 aa, Artificial Sequence
  Human antibody library
VIYPSGGFTHYADSVKG

SEQ ID NO: 234 - protein, 6 aa, Artificial Sequence
  Human antibody library
YPSGGF

SEQ ID NO: 235 - protein, 11 aa, Artificial Sequence
  Human antibody library
GRKTTVTDFDY

SEQ ID NO: 236 - protein, 120 aa, Artificial Sequence
  Human antibody library
EVQLLESGGGLVQPGGSLRLSCAASGFTFSIYSMNWVRQAPGKGLEWVSVIYPSGGFTHYADSVKGRFTISRDNSKNTLYLQMNSLRAEDTAVYYCARGRKTTVTDFDYWGQGTLVTVSS

SEQ ID NO: 237 - protein, 14 aa, Artificial Sequence
  Human antibody library
TGTSSDVGGYNYVS

SEQ ID NO: 238 - protein, 7 aa, Artificial Sequence
  Human antibody library
EVSHRPS

SEQ ID NO: 239 - protein, 10 aa, Artificial Sequence
  Human antibody library
SSYTSGSTRV

SEQ ID NO: 240 - protein, 113 aa, Artificial Sequence
  Human antibody library
QSALTQPASVSGSPGQSITISCTGTSSDVGGYNYVSWYQQHPGKAPKVMIYEVSHRPSGVSNRFSGSKSGNTASLTISGLQAEDEADYYCSSYTSGSTRVFGGGTKVTVLGQP

SEQ ID NO: 241 - protein, 5 aa, Artificial Sequence
  Human antibody library
MYMMM

SEQ ID NO: 242 - protein, 7 aa, Artificial Sequence
  Human antibody library
GFTFSMY

SEQ ID NO: 243 - protein, 17 aa, Artificial Sequence
  Human antibody library
SIYPSGGITFYADSVKG

SEQ ID NO: 244 - protein, 6 aa, Artificial Sequence
  Human antibody library
YPSGGI

SEQ ID NO: 245 - protein, 11 aa, Artificial Sequence
  Human antibody library
IKLGTVTTVDY

SEQ ID NO: 246 - protein, 120 aa, Artificial Sequence
  Human antibody library
EVQLLESGGGLVQPGGSLRLSCAASGFTFSMYMMMWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 247 - protein, 14 aa, Artificial Sequence
  Human antibody library
TGTSSDVGAYNYVS

SEQ ID NO: 248 - protein, 7 aa, Artificial Sequence
  Human antibody library
DVSNRPS

SEQ ID NO: 249 - protein, 10 aa, Artificial Sequence
  Human antibody library
SSYTSSSTRV

SEQ ID NO: 250 - protein, 113 aa, Artificial Sequence
  Human antibody library
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVLGQP

SEQ ID NO: 251 - protein, 5 aa, Artificial Sequence
  Human antibody library
AYRMV

SEQ ID NO: 252 - protein, 7 aa, Artificial Sequence
  Human antibody library
GFTFSAY

SEQ ID NO: 253 - protein, 17 aa, Artificial Sequence
  Human antibody library
RIYPSGGFTFYADSVKG

SEQ ID NO: 254 - protein, 6 aa, Artificial Sequence
  Human antibody library
YPSGGF

SEQ ID NO: 255 - protein, 10 aa, Artificial Sequence
  Human antibody library
ALGPLSPLDS

SEQ ID NO: 256 - protein, 119 aa, Artificial Sequence
  Human antibody library
EVQLLESGGGLVQPGGSLRLSCAASGFTFSAYRMVWVRQAPGKGLEWVSRIYPSGGFTFYADSVKGRFTISRDNSKNTLYLQMNSLRAEDTAVYYCARALGPLSPLDSWGQGTLVTVSS

SEQ ID NO: 257 - protein, 14 aa, Artificial Sequence
  Human antibody library
TGTSSDVGGYNYVS

SEQ ID NO: 258 - protein, 7 aa, Artificial Sequence
  Human antibody library
EVSNRPS

SEQ ID NO: 259 - protein, 10 aa, Artificial Sequence
  Human antibody library
SSYRSGNTLV

SEQ ID NO: 260 - protein, 113 aa, Artificial Sequence
  Human antibody library
QSELTQPASVSGSPGQSITISCTGTSSDVGGYNYVSWYQQHPGKAPKLMIYEVSNRPSGVPDRFSGSKSGNTASLTISGLQAEDEGDYYCSSYRSGNTLVFGGGTKVTVLGQP

SEQ ID NO: 261 - protein, 209 aa, Artificial Sequence
  SD15 fusion protein
  DOMAIN (1)..(61): human ILL-15Ra Sushi domain (NM-002189)
  DOMAIN (62)..(75): IRD-11exone3
  DOMAIN (76)..(95): linker
  DOMAIN (96)..(209): human IL-15 (NM-000585-3)
CPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 262 - protein, 659 aa, Artificial Sequence
  Fusion protein
EVQLLESGGGLVQPGGSLRLSCAASGFTFSMYMMMWVRQAPGKGLEWVSSIYPSGGITFYADSVKGRFTISRDNSKNTLYLQMNSLRAEDTAIYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 263 - protein, 659 aa, Artificial Sequence
  Fusion protein
EVQLLESGGGLVQPGGSLRLSCAASGFTFSMYMMMWVRQAPGKGLEWVSSIYPSGGITFYADSVKGRFTISRDNSKNTLYLQMNSLRAEDTAIYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 264 - protein, 5 aa, Artificial Sequence
  synthetic
AYAMA

SEQ ID NO: 265 - protein, 120 aa, Artificial Sequence
  synthetic
EVQLLESGGGLVQPGGSLRLSCAASGFTFSAYAMAWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 266 - protein, 5 aa, Artificial Sequence
  synthetic
AYRMF

SEQ ID NO: 267 - protein, 120 aa, Artificial Sequence
  synthetic
EVQLLESGGGLVQPGGSLRLSCAASGFTFSAYRMFWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 268 - protein, 5 aa, Artificial Sequence
  synthetic
AYLMV

SEQ ID NO: 269 - protein, 120 aa, Artificial Sequence
  synthetic
EVQLLESGGGLVQPGGSLRLSCAASGFTFSAYLMVWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 270 - protein, 5 aa, Artificial Sequence
  synthetic
AYVMF

SEQ ID NO: 271 - protein, 120 aa, Artificial Sequence
  synthetic
EVQLLESGGGLVQPGGSLRLSCAASGFTFSAYVMFWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 272 - protein, 5 aa, Artificial Sequence
  synthetic
AYVMS

SEQ ID NO: 273 - protein, 120 aa, Artificial Sequence
  synthetic
EVQLLESGGGLVQPGGSLRLSCAASGFTFSAYVMSWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 274 - protein, 5 aa, Artificial Sequence
  synthetic
GYLMV

SEQ ID NO: 275 - protein, 120 aa, Artificial Sequence
  synthetic
EVQLLESGGGLVQPGGSLRLSCAASGFTFSGYLMVWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 276 - protein, 5 aa, Artificial Sequence
  synthetic
GYQML

SEQ ID NO: 277 - protein, 120 aa, Artificial Sequence
  synthetic
EVQLLESGGGLVQPGGSLRLSCAASGFTFSGYQMLWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 278 - protein, 5 aa, Artificial Sequence
  synthetic
GYSMF

SEQ ID NO: 279 - protein, 120 aa, Artificial Sequence
  synthetic
EVQLLESGGGLVQPGGSLRLSCAASGFTFSGYSMFWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 280 - protein, 5 aa, Artificial Sequence
  synthetic
GYWMA

SEQ ID NO: 281 - protein, 120 aa, Artificial Sequence
  synthetic
EVQLLESGGGLVQPGGSLRLSCAASGFTFSGYWMAWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 282 - protein, 5 aa, Artificial Sequence
  synthetic
QYLMY

SEQ ID NO: 283 - protein, 120 aa, Artificial Sequence
  synthetic
EVQLLESGGGLVQPGGSLRLSCAASGFTFSQYLMYWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 284 - protein, 5 aa, Artificial Sequence
  synthetic
QYVMF

SEQ ID NO: 285 - protein, 120 aa, Artificial Sequence
  synthetic
EVQLLESGGGLVQPGGSLRLSCAASGFTFSQYVMFWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 286 - protein, 5 aa, Artificial Sequence
  synthetic
QYYMY

SEQ ID NO: 287 - protein, 120 aa, Artificial Sequence
  synthetic
EVQLLESGGGLVQPGGSLRLSCAASGFTFSQYYMYWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 288 - protein, 5 aa, Artificial Sequence
  synthetic
SYLMS

SEQ ID NO: 289 - protein, 120 aa, Artificial Sequence
  synthetic
EVQLLESGGGLVQPGGSLRLSCAASGFTFSSYLMSWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 290 - protein, 5 aa, Artificial Sequence
  synthetic
SYLMV

SEQ ID NO: 291 - protein, 120 aa, Artificial Sequence
  synthetic
EVQLLESGGGLVQPGGSLRLSCAASGFTFSSYLMVWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 292 - protein, 12 aa, Artificial Sequence
  synthetic
SYLMTTCCLDHK

SEQ ID NO: 293 - protein, 120 aa, Artificial Sequence
  synthetic
EVQLLESGGGLVQPGGSLRLSCAASGFTFSSYLMTWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 294 - protein, 5 aa, Artificial Sequence
  synthetic
SYQMV

SEQ ID NO: 295 - protein, 120 aa, Artificial Sequence
  synthetic
EVQLLESGGGLVQPGGSLRLSCAASGFTFSSYQMVWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 296 - protein, 5 aa, Artificial Sequence
  synthetic
SYSMA

SEQ ID NO: 297 - protein, 120 aa, Artificial Sequence
  synthetic
EVQLLESGGGLVQPGGSLRLSCAASGFTFSSYSMAWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 298 - protein, 5 aa, Artificial Sequence
  synthetic
SYVMF

SEQ ID NO: 299 - protein, 120 aa, Artificial Sequence
  synthetic
EVQLLESGGGLVQPGGSLRLSCAASGFTFSSYVMFWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 300 - protein, 5 aa, Artificial Sequence
  synthetic
SYVMS

SEQ ID NO: 301 - protein, 120 aa, Artificial Sequence
  synthetic
EVQLLESGGGLVQPGGSLRLSCAASGFTFSSYVMSWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 302 - protein, 5 aa, Artificial Sequence
  synthetic
SYVMY

SEQ ID NO: 303 - protein, 120 aa, Artificial Sequence
  synthetic
EVQLLESGGGLVQPGGSLRLSCAASGFTFSSYVMYWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 304 - protein, 5 aa, Artificial Sequence
  synthetic
SYYMF

SEQ ID NO: 305 - protein, 120 aa, Artificial Sequence
  synthetic
EVQLLESGGGLVQPGGSLRLSCAASGFTFSSYYMFWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 306 - protein, 5 aa, Artificial Sequence
  synthetic
SYYMV

SEQ ID NO: 307 - protein, 120 aa, Artificial Sequence
  synthetic
EVQLLESGGGLVQPGGSLRLSCAASGFTFSSYYMVWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 308 - protein, 5 aa, Artificial Sequence
  synthetic
YYSMV

SEQ ID NO: 309 - protein, 120 aa, Artificial Sequence
  synthetic
EVQLLESGGGLVQPGGSLRLSCAASGFTFSYYSMVWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 310 - protein, 5 aa, Artificial Sequence
  synthetic
WYLMA

SEQ ID NO: 311 - protein, 120 aa, Artificial Sequence
  synthetic
EVQLLESGGGLVQPGGSLRLSCAASGFTFSWYLMAWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 312 - protein, 5 aa, Artificial Sequence
  synthetic
WYQMS

SEQ ID NO: 313 - protein, 120 aa, Artificial Sequence
  synthetic
EVQLLESGGGLVQPGGSLRLSCAASGFTFSWYQMSWVRQAPGKGLEWVSSIYPSGGITFYADSVKGRFTISRDNSKNTLYLQMNSLRAEDTAIYYCARIKLGTVTTVDYWGQGTLVTVSS

SEQ ID NO: 314 - protein, 20 aa, Artificial Sequence
  synthetic
SGGSGGGGSGGGSGGGGSLQ

SEQ ID NO: 315 - protein, 198 aa, Artificial Sequence
  synthetic
  DOMAIN (1)..(61): human IL-15Ra Sushi domain (NM-002189)
  DOMAIN (62)..(64): IRD-11exone3
  DOMAIN (65)..(84): linker1
  DOMAIN (85)..(198): human IL-15 (NM-000585-3)
CPPPMSVEHADIWVKSYSLYSRERYICNSGFKRKAGTSSLTECVLNKATNVAHWTTPSLKCIRDSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 316 - protein, 25 aa, Artificial Sequence
  synthetic
SGGGGSGGGGSGGGGSGGGGSGGGG

SEQ ID NO: 317 - protein, 203 aa, Artificial Sequence
  synthetic
  DOMAIN (1)..(61): human ILL-15Ra Sushi domain (NM-002189)
  DOMAIN (62)..(64): human IL-15Ra Sushi domain (NM-002189) IRD-11exone3
  DOMAIN (65)..(89): linker2
  DOMAIN (90)..(203): human IL-15 (NM-000585-3)
CPPPMSVEHADIWVKSYSLYSRERYICNSGFKRKAGTSSLTECVLNKATNVAHWTTPSLKCIRDSGGGGSGGGGSGGGGSGGGGSGGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 318 - protein, 30 aa, Artificial Sequence
  synthetic
SGGGGSGGGGSGGGGSGGGGSGGGGSGGGG

SEQ ID NO: 319 - protein, 208 aa, Artificial Sequence
  synthetic
  DOMAIN (1)..(61): human ILL-15Ra Sushi domain (NM-002189)
  DOMAIN (62)..(64): IRD-11exone3
  DOMAIN (65)..(94): linker3
  DOMAIN (95)..(208): human IL-15 (NM-000585-3)
CPPPMSVEHADIWVKSYSLYSRERYICNSGFKRKAGTSSLTECVLNKATNVAHWTTPSLKCIRDSGGGGSGGGGSGGGGSGGGGSGGGGSGGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 320 - protein, 40 aa, Artificial Sequence
  synthetic
SGGGGSGGGGSGGGGSGGGGSGGGGSGGGGSGGGGSGGGG

SEQ ID NO: 321 - protein, 218 aa, Artificial Sequence
  synthetic
  DOMAIN (1)..(61): human ILL-15Ra Sushi domain (NM-002189)
  DOMAIN (62)..(64): IRD-11exone3
  DOMAIN (65)..(104): linker4
  DOMAIN (105)..(218): human IL-15 (NM-000585-3)
CPPPMSVEHADIWVKSYSLYSRERYICNSGFKRKAGTSSLTECVLNKATNVAHWTTPSLKCIRDSGGGGSGGGGSGGGGSGGGGSGGGGSGGGGSGGGGSGGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 322 - protein, 50 aa, Artificial Sequence
  synthetic
SGGGGSGGGGSGGGGSGGGGSGGGGSGGGGSGGGGSGGGGSGGGGSGGGG

SEQ ID NO: 323 - protein, 228 aa, Artificial Sequence
  synthetic
  DOMAIN (1)..(61): human ILL-15Ra Sushi domain (NM-002189)
  DOMAIN (62)..(64): IRD-11exone3
  DOMAIN (65)..(114): linker5
  DOMAIN (115)..(228): human IL-15 (NM-000585-3)
CPPPMSVEHADIWVKSYSLYSRERYICNSGFKRKAGTSSLTECVLNKATNVAHWTTPSLKCIRDSGGGGSGGGGSGGGGSGGGGSGGGGSGGGGSGGGGSGGGGSGGGGSGGGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 324 - protein, 18 aa, Artificial Sequence
  synthetic
SGGSGGGGSGGGSGGGGS

SEQ ID NO: 325 - protein, 646 aa, Artificial Sequence
  synthetic
  DOMAIN (1)..(120): tccLD7-102 heavy chain variable domain
  DOMAIN (121)..(449): IgG1 CH1-CH2-CH3 domain
  DOMAIN (450)..(646): deSD15-20
EVQLLESGGGLVQPGGSLRLSCAASGFTFSAYRMFWVRQAPGKGLEWVSSIYPSGGITFYADSVKGRFTISRDNSKNTLYLQMNSLRAEDTAIYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSCPPPMSVEHADIWVKSYSLYSRERYICNSGFKRKAGTSSLTECVLNKATNVAHWTTPSLKCIRDSGGSGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 326 - protein, 216 aa, Artificial Sequence
  synthetic
  DOMAIN (1)..(113): VL
  DOMAIN (114)..(216): CL
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 327 - protein, 215 aa, Artificial Sequence
  synthetic
  DOMAIN (1)..(113): VL
  DOMAIN (114)..(215): CL w/ C-terminal serine deleted
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 328 - protein, 5 aa, Artificial Sequence
  synthetic
  VARIANT (1)..(1): residue is A G M Q S Y or W
  VARIANT (3)..(3): residue is A L M Q R S V W or Y
  VARIANT (5)..(5): residue is A F L M S T V or Y
XYXMX

(19) United States
(12) Patent Application Publication
(10) Pub. No.: US 2024/0059775 A9
(43) Pub. Date: Feb. 22, 2024

(54) IMMUNOMODULATORY AGENTS

(71) Applicant: KADMON CORPORATION, LLC, Bridgewater, NJ (US)

(72) Inventors: Samuel D. Waksal, New York, NY (US); Zhenping Zhu, Woodcliff Lake, NJ (US); Yan Wu, Doylestown, PA (US); Stella A. Martomo, Edgewater, NJ (US); Zhaojing Zhong, Bronx, NY (US); Dan Lu, Montvale, NJ (US)

(21) Appl. No.: 16/812,867

(22) Filed: Mar. 9, 2020

Related U.S. Application Data
(63) Continuation of application No. 16/529,373, filed on Aug. 1, 2019, now abandoned.
Application No. 16/529,373 is a continuation of application No. 15/111,102, filed on Jul. 12, 2016, now Pat. No. 10,407,502.
(60) Provisional application No. 61/927,907, filed on Jan. 15, 2014.

(51) Int. Cl. A61B 17/70

(52) U.S. Cl. CPC A61B 17/7037; A61B 17/7035

(57) ABSTRACT

The invention provides antibodies that specifically bind to PD-L1 and fusion molecules comprising PD-L1 binding proteins constructed with an IL 15 receptor-binding domain, nucleic acid molecules encoding the same, and therapeutic compositions thereof. The agents inhibit PD-L1-mediated immunosuppression and enhance cell and cytokine mediated immunity for the treatment of neoplastic and infectious diseases.

### CROSS-REFERENCE TO RELATED APPLICATIONS

This application is a continuation of U.S. Ser. No.: 16/529,373, filed Aug. 1, 2019, which is a continuation of U.S. Ser. No.: 15/111,102, filed Jul. 12, 2016, now U.S. Pat. No.: 10,407,502, issued Sep. 10, 2019, which is a 371 of PCT/US2015/011657, filed Jan. 15, 2015, which claims the benefit of priority to U.S. Provisional Application No. 61/927,907, filed Jan. 15, 2014, the contents of which are incorporated herein by reference in their entireties.

### FIELD OF THE INVENTION

The invention provides monoclonal antibodies that specifically bind to PD-L1 and bispecific fusion molecules comprising PD-L1 binding proteins constructed with IL15 and an IL15 receptor alpha sushi domain, nucleic acid molecules encoding the same, and therapeutic compositions thereof. The agents enhance T cell and NK cell function to increase disorders including cancers and infectious diseases.

### BACKGROUND OF THE INVENTION

Programmed death 1 (PD-1) is a member of the CD28 family of receptors comprising CD28, CTLA-4, PD-1, ICOS, and BTLA (Freeman et al. (2000) J Exp Med 192:1027-34; Latchman et al. (2001) Nat Immunol 2:261-8). PD-1 is an inducible immunosuppressive receptor mainly upregulated on activated T cells and B cells during the progression of immunopathological conditions. PD-1 interaction with its ligand PD-L1 results in the inhibition of TCR and BCR mediated proliferation and cytokine production and induction of apoptosis of antigen specific T cells through the intrinsic PD-1 mediated negative signaling of an immunoreceptor tyrosine-based inhibitory motif (ITIM) (Agata et al. (1996) Int. Immunol. 8:765, Unkeless and Jin. (1997) Curr. Opin. Immunol. 9:338-343, Okzaki et al. (2001) PNAS 98:13866-71, Dong et al. (2002) Nat. Med. 8:793-800). PD-L1 is a cell surface glycoprotein and a major ligand for PD-1. PD-L1 is also inducible on lymphoid tissues and non-lymphoid peripheral tissues following cellular activation. PD-L1 is upregulated in a variety of affected cell types including cancer and stromal cells in addition to immune cells, and plays an active role in immunosuppression during the course of the deterioration of diseases (Iwai et al (2002) PNAS 99:12293-7, Ohigashi et al. (2005) Clin Cancer Res 11:2947-53). PD-L1 upregulation has been linked to poor clinical outcomes in a variety of cancers and viral infection (Hofmeyer et al. (2011) J. BioMed. Biotech. 2011:1-9, McDermott and Atkins. (2013) Cancer Med. 2:662-73). The blockade of PD-1 or PD-L1 by antibody promoted CD8 T cell infiltration, CTL activity and increased presence of Th1 cytokine IFN-gamma in preclinical and clinical settings (Zhou et al. (2010) J. Immunol. 185:5082-92, Nomi et al. (2007) Clin Cancer Res. 13:2152-7, Flies et al. (2011) Yale J. Bio. Med. 48:409-21, Zitvogel and Kroemer. (2012) OncoImmunol. 1:1223-25). PD-L1 antibody as an immunomodulating agent has been shown to be efficacious when used as monotherapy or combined with antibodies to other immunosuppressive molecules.

However, the immunomodulating intervention to immunosuppressive factors only partially resolves the problems associated with impaired immunity in cancer, infection, and other diseases. It is still highly desirable to utilize biotherapeutic agents to directly stimulate and expand effector immune cells for lifting weakened innate and adaptive immune response to a more effective level to control tumor and infection. Immunotherapy using cytokines including interleukins, i.e. IL-2, IL-12, IL15, IL-21, and TNFα, GM-CSF, etc., has been shown to be efficacious to some extent in the treatment of cancer and infection, but clinical outcome is often limited by systemic toxicity associated with the high blood concentrations of cytokine that need to obtain efficacy and lack of specificity of target in affected cells and tissues.

Among assessed cytokines, IL15 has been recognized to be dedicated to stimulate effector and central memory CD8 T cells composing of a subset of antigen specific CD8 cells to exert antitumor immunity without modulating effects on other T cell populations. Moreover, unlike IL-2 that activates Treg, IL15 has been shown to have capacity to rescue T cells from apoptosis induced by Treg and other immunosuppressive cells in addition to its ability to activate natural killer (NK) cells and effector and memory CD8 T cells (Van Belle et al. (2012) PLoS One 7:e45299, Obar and Lefrancois. (2010) J. Immuno1.185:263-72, Pelletier and Girard. (2006) J Immunol 177:100-108, Elpek et al. (2010) PNAS 107:21647-21652).

IL15 was identified as a γc cytokine in 1994 based on its ability to stimulate the proliferation of the murine T cell line CTLL-2 (Grabstein et al. (1994) Science 264:965-8, Bamford et al. (1996) PNAS 93:2897-902). Human IL15 shares approximately 97% and 96% amino acid sequence identity with simian and cynomolgus IL15, respectively. Human and mouse IL15 have 73% homology and are comparably active on mouse cells. IL15 is a 12.5 KD protein (114 amino acids), secreted by DC, macrophage and granular cells as a 14-15 kDa glycoprotein, and also a member of the four α-helix bundle-containing cytokines (Andderson et al. (1995) J Biol Chem. 270:29862-9, Steel et al. (2012) Trends Pharmacol. Sci. 33:35-41). IL15 is typically formed a complex with IL15 receptor alpha expressed on APCs prior to binding to functional IL15 receptor beta and gamma units on T cells and NK cells. IL15 may be presented in trans to responsive cells expressing CD122 and CD132 by cells expressing the cytokine itself bound to a membrane form of the receptor alpha chain (Dubois et al. (2002) Immunity 17:537-47). IL15 receptor alpha sushi domain (29.5KD in size) is a critical component to form a complex with IL15 prior to properly engagement with receptor β and γ (Wei et al. (2001) J. Immuno1.167:277-82). IL15 and IL15Rα complex and IL15/IL15Rα sushi domain fusion protein were reported to be highly potent to stimulate CD8 T cells and NK cells in vitro and in vivo compared to IL15 alone (Mortier et al. (2005) J Biol Chem. 281:1612-19, Stoklasek et al. (2006) J. Immunol. 177:6072-80). IL15 also induces the proliferation and differentiation of stimulated human B cells (Armitage et al. (1995) J Immunol. 154:483-90). It was suggested that IL15 mostly opposed activation-induced cell death (AICD) by acting to prolong the survival of T lymphocytes (Marks-Konczalik et al. (2000) PNAS 97:11445-50). IL15 has an exceptional ability to support the maintenance of NK cells and memory phenotype and antigen specific memory CD8 T cells (Ma et al. (2006) Annu Rev Immunol. 24:657-79). Thus, among most active cytokines in immunomodulation, IL15 has an unique capacity to mediate many important aspects of immunity against a variety of tumor types and viral infection including HIV, HBV, HCV, LCMV, etc (Steel et al. (2012) Trends Pharmacol. Sci. 33:35-41, Verbist and Klonowski, (2012) Cytokine. 59:467-478).

### SUMMARY OF THE INVENTION

The present invention provides antibodies and binding proteins that bind to PD-L1. In certain embodiments of the invention, the antibodies bind to PD-L1 and block interaction with PD-1. By blocking the interaction of PD-L1 with PD-1, such antibodies are useful to reduce or inhibit immunosuppression.

In another aspect, the invention provides antibodies and binding proteins that bind specifically to PD-L1 and at least one other molecule. Examples of such embodiments include PD-L1 binding proteins that also bind to one or more other ligands and/or receptors, which may be membrane bound or soluble.

In another aspect, the invention provides molecules, such as fusion proteins that bind PD-L1 that, apart from reducing or inhibiting immunosuppression by binding to PD-L1, also promote one or more immune responses by interaction with other ligands or receptors. In an embodiment of the invention, the molecule binds to PD-L1 on target cells, and also stimulates a cell-mediated immune response, for example, by promoting proliferation of T cells and/or NK cells. In an embodiment of the invention, the molecule stimulates cells that respond to an interleukin or an interferon, such as, without limitation, IL2, IL7, IL15, and IL21. In an embodiment of the invention, the molecule includes a sequence or domain that promotes IL15 stimulation of the IL15 receptor (IL15R). In an embodiment of the invention, the molecule that promotes IL15R stimulation is a portion of the IL15R alpha chain comprising a sushi domain. In an embodiment of the invention, the molecule provides the sushi domain of the IL15R alpha chain. In an embodiment of the invention, the molecule provides a complex of IL15 and the sushi domain of the IL15R alpha chain, which may be covalent or non-covalent. The experiments disclosed herein demonstrate that single molecules containing both a PD-L1binding domain that blocks binding or PD-L1 to PD-1, and an IL15R stimulating domain, promote a better immune response than separate molecules used together. More particularly, providing a molecule that provides an anti-PD-L1 antibody domain as well as a hybrid domain comprising IL15 and the IL15 alpha chain sushi domain, promoted increased proliferation, Th1 cytokine release, and killing activity-related molecules of NK and T cells, compared to providing the domains in separate molecules.

In one embodiment, the invention provides an antibody or fragment that binds to PD-L1, which comprises a heavy chain CDR-1H which has the sequence X₁YX₂MX₃ (SEQ ID NO:328) wherein X₁ is A, G, M, Q, S, Y, or W, X₂ is A, L, M, Q, R, S, V, W, or Y, and X₃ is A, F, L, M, S, T, V, or Y, a heavy chain CDR-2H which has SEQ ID NO:243, and a heavy chain CDR-3H which has the sequence of SEQ ID NO:245. In certain such embodiments, the heavy chain CDR-1H has a sequence selected from SEQ ID NO:241, SEQ ID NO:264, SEQ ID NO:266, SEQ ID NO:268, SEQ ID NO:270, SEQ ID NO:272, SEQ ID NO:274, SEQ ID NO:276, SEQ ID NO:278, SEQ ID NO:280, SEQ ID NO:282, SEQ ID NO:284, SEQ ID NO:286, SEQ ID NO:288, SEQ ID NO:290, SEQ ID NO:292, SEQ ID NO:294, SEQ ID NO:296, SEQ ID NO:298, SEQ ID NO:300, SEQ ID NO:302, SEQ ID NO:304, SEQ ID NO:306, SEQ ID NO:308, SEQ ID NO:310, and SEQ ID NO:312. In such embodiments, the heavy chain variable domain is at least 80%, or at least 85%, or at least 90%, or at least 95% identical to SEQ ID NO:246. The antibodies may further comprise a light chain variable domain which comprises a CDR-1L which has SEQ ID NO:247, a CDR-2L which has SEQ ID NO:248, and a CDR-3L which has SEQ ID NO:249. In some such embodiments the light chain variable domain is at least 80%, or at least 85%, or at least 90%, or at least 95% identical to SEQ ID NO:250. In another embodiment, the invention provides an antibody or fragment thereof that binds to PD-L1, wherein the light chain comprises a CDR-1L which has SEQ ID NO:247, a CDR-2L which has SEQ ID NO:248, and a CDR-3L which has SEQ ID NO:249.

The invention also provides conjugates of the antibodies, for example, and without limitation, to imaging agents, therapeutic agents, or cytotoxic agents.

The invention further provides compositions comprising the antibodies and conjugates and a pharamaceutically acceptable carrier.

In another aspect, the invention provides a fusion protein capable of binding to PDL1, which also stimulates an immune response mediated by, for example, a T cell or an NK cell. In an embodiment of the invention, the fusion protein includes a portion that binds to IL15 receptor. In other embodiments, the fusion protein includes a portion that binds to, e.g., an interleukin receptor or an interferon receptor. In an embodiment of the invention, the portion of the fusion protein that binds to PD-L1 is an antibody or PD-L1 binding fragment thereof. In an embodiment of the invention, the IL15 receptor-binding portion is IL15, whose binding may be enhanced by the presence in the fusion protein of an IL15R alpha sushi domain.

The invention provides a method of inhibiting the interaction of PD1 with PD-L1 in a subject, which comprises administering an effective amount of an antibody or fragment of the invention. The invention further provides a method of inhibiting immunosuppression mediated by PD-L1 which comprises administering an effective amount of the antibody or fragment of the invention, or a fusion protein of the invention.

The invention further provides a method of stimulating an immune response against a cell or tissue that expresses PD-L1, which comprises administering to a subject an effective amount of the antibody or fragment of the invention, or a fusion protein of the invention. In certain embodiments, the cell or tissue the expresses PD-L1 is a neoplastic cell or an infected cell.

### BRIEF DESCRIPTION OF THE FIGURES

FIG. 1 depicts binding to human hPDL1-Fc (top left panel), blocking of hPDL1 to hPD1 (bottom left panel), binding to mouse mPDL1-Fc (top right panel), and blocking of mPDL1 to hPD1 (bottom right panel) of antibodies tccR3λF8, tccR3κA11, tccR3λH4, tctR3κA8, sR3λD7, and R2κA6.

FIG. 2 depicts binding to human hPDL1-Fc (top left panel), blocking of hPDL1 to hPD1 (bottom left panel), binding to mouse mPDL1-Fc (top right panel), and blocking of mPDL1 to hPD1 (bottom right panel) of antibodies sR3λD7, tccR3κB7, tccR3κA4, tccR3λF8, tccR3λH7, tccR3λH4, and tccR3κD9.

FIG. 3 depicts binding to human hPDL1-Fc (top left panel), blocking of hPDL1 to hPD1 (bottom left panel), binding to mouse mPDL1-Fc (top right panel), and blocking of mPDL1 to hPD1 (bottom right panel) of antibodies tccR3κF8, tccR3κD9, tccR3λD7, tccR3λD7 sR3κF10, sR3λD7, and tccR3λF8.

FIG. 4 depicts binding to human hPDL1-Fc (top left panel), blocking of hPDL1 to hPD1 (bottom left panel), binding to mouse mPDL1-Fc (top right panel), and blocking of mPDL1 to hPD1 (bottom right panel) of antibodies R2κA6, sR3λD7, tccR3λD7, tccR3κB7, and tccR3κH4.

FIG. 5 depicts binding to PDLI-293 cells (top) and MDS-MB-231 (bottom) cells of antibodies sR3λD7, tctR3κA8, tccR3κA11, tccR3λD7, tccR3κD9, tccR3λF8, tccR3κF8, tccR3κF10, tccR3λH4, tccR3κB7, and tccR3κA4.

FIG. 6 depicts anti-PD-L1 antibodies binding to (A) human monocyte-derived dendritic cells, (B) human cancer cell line expressing PD-L1 MDA-MB-231 cells, and (C) mouse cell line expressing PD-L1 B16-F10.

FIG. 7 shows functional blocking activity of anti-PD-L1 antibodies measured by (A) increase in CD4 proliferation when activated by aCD3 and PD-L1Fc coated beads, (B) increase in cytokine secretion in SEB-activated human PBMC, and (C) increase in CD4 proliferation in Mixed-Lymphocyte Reaction with mo-DC.

FIG. 8 shows CD4 and CD8 activation when both anti-PDL1 antibody and IL15 were present in (A) mixed-lymphocyte reaction with mo-DC, and (B) CD8 stimulation by αCD3 and PD-L1Fc coated beads.

FIG. 9 shows anti-PD-L1-sushi domain-IL15 (termed anti-PDL1-SD15) fusion proteins retain binding to PD-L1 as measured by (A) solid-phase ELISA, and (B) binding to CD4 activated by aCD3 coated beads.

FIG. 10 shows PBMC cultured in vitro with anti-PD-L1-SD15 fusion proteins resulted in increased NK cell number (A), increased CD8 cell number (B) and activation status as measured by % granzymeB (C). No effect was observed on CD4 cells (D).

FIG. 11 shows anti-PD-L1-SD15 fusion proteins function to activate CD8 similarly to IL15 when added to in vitro CD8 stimulation in the presence of aCD3 coated beads (A). However, in the presence of aCD3 and PD-L1Fc coated beads, anti-PD-L1-SD15 fusion proteins can increase CD8 proliferation by more than five-fold when compared to IL15 (B). cD7-SD15neg is anti-PD-L1 cD7 with non-functional IL15 serving as negative controls.

FIG. 12 shows CD8 activation in the presence of PD-L1Fc on the antigen presenting cells. Anti-PD-L1-SD15 fusion protein cD7-SD15 stimulated CD8 at significantly lower concentrations as measured by (A) percent increase in granzymeB positive CD8, and (B) increased in total cytokine secretions. The maximum levels of CD8 activation were also increased in CD8 activated by aCD3 and PD-L1Fc with addition of cD7-SD15 as compared to IL15. (C) Data on CD8 proliferation in the presence of both anti-PD-L1 antibody and free IL15 (dotted lines) is superimposed on data of CD8 proliferation in presence of anti-PD-L1-SD15 fusion protein (straight lines).

FIGS. 13A1-13B3 provides amino acid sequences of V_{H} (FIG. 13A1, FIG. 13A2, and FIG. 13A3) and V_{L} (FIG. 13B1, FIG. 13B2, and FIG. 13B3) chains of anti-PD-L1 antibodies. For V_{H} sequences, boxed regions indicate CDRs. For CDR-1H, Chothia CDRs are in italics, and Kabat CDRs are underlined. For CDR-2H, Kabat CDRs are coextensive with the boxed sequences, with Chothia CDRs initalics. For V_{L} sequences, boxed regions indicate Kabat/Chothia CDRs.

FIG. 14 shows the amino acid sequences of SD15 (SEQ ID NO:261), which includes the IL15R alpha sushi domain and IL15, tccλD7HC-SD15 (SEQ ID NO:262) and the LALA mutant of tccλD7HC-SD15 (SEQ ID NO:263), which contains alanine substitutions for two adjacent leucines at positions (Leu²³⁴ and Leu²³⁵) in the heavy chain constant region important for FcγRI.

FIG. 15 shows cytoxicity of anti-PD-L1-SD15 fusion protein (CD7SD15) compared to the PD-L1 binding portion of the molecule alone (cD7) and a fusion protein containing a binding domain specific for KLH and the IL15 domain (KLHSD15). Human CD8 T cells and MAD-MB-231 tumor cells were co-cultured in IMDM supplemented with 10% FBS for 7 days. Tumor cell killing activity was assessed by the measurement of the number of dead tumor cells stained by Viability Dye eFluor 780 in FACS.

FIG. 16 shows anti-PD-L1-SD15 fusion protein prolonged the survival rate of mice bearing PD-L1 expressing tumors. Balb/c mice were intravenously injected with 2×10⁵ murine CT26 colon tumor cells. 24 hrs later, mice received i.p. administration of the anti-PD-L1 antibody cD7 (purple line:75μg per dose), anti-PD-L1-SD15 fusion protein cD7-SD15 (green line:75μg per dose, blue line: 25μg per dose) or sD7-SD15 (Grey line:75μg per dose, Red line: 25μg per dose) twice a week in the first week then once weekly in the rest of treatment course. Mice in control groups received an equal volume of saline or normal IgG solution. Survival rate was measured by using Kaplan-Meier Plot.

FIG. 17 shows binding of two affinity matured anti-PDL1 antibdodies to soluble human PDL1, soluble mouse PDL1, and soluble rat PDL1, and no binding to human PDL2.

FIG. 18 shows blocking of human PD1 to human PDL1 (left panel) and blocking of mouse PD1 to mouse PDL1 (right panel) by two affinity matured anti-PDL1 antibodies, compared to their parent tcc λD7 antibody.

FIG. 19 shows two affinity matured variants of anti-PD-L1 antibodies tcc2D7 have higher binding activity to PD-L1 expressing human MDA-MB-231 tumor cells as measured by flow cytometry.

FIG. 20 shows affinity matured variants of anti-PD-L1 antibodies tccλD7 having increased potency to promote production of Th1 cytokines IL2 (top panel) and IFNγ (bottom panel).

FIG. 21 shows binding of fusion proteins of the invention to PD-L1-expressing MDA-MB-231 tumor cells.

FIG. 22 shows stimulatory activity of proteins of the invention on IL15-responsive human megakaryoblastic leukemia cells.

FIG. 23 shows hPD-L1 binding (left panel) and ligand blocking (right panel) activity for fusion proteins of the invention.

FIG. 24 shows the results of size exclusion chromatography for fusion proteins of the invention.

FIG. 25 shows serum stability for fusion proteins of the invention.

### DETAILED DESCRIPTION

The interaction of PD-1 on immune cells with PD-L1 inhibits proliferation and cytokine production by immune cells. PD-L1 is also inducible and upregulated in various tissues, including cancer. Together, PD-1 and PD-L1 play a role in immunosuppression. The invention provides novel antibodies or antigen binding fragments of such antibodies that bind to PD-L1 and block the interaction with PD-1. In embodiments of the invention, the antibodies reduce or inhibit immunosuppression.

Novel antibodies of the invention are set forth in Table 1 and the accompanying sequence listing, which set forth amino acid sequences of heavy and light chain CDRs (identified according to the identification systems of Kabat and Chothia), as well as complete heavy and light chain variable region. The first two heavy chain CDRs are identified according to the common systems of Kabat and Chothia, which provide distinct, but overlapping locations for the CDRs. A comparison of the numerous heavy and light chains shows a significant similarity among many of the CDR sequences. Accordingly, it would be expected that many of the CDRs can be mixed and matched among the sequences.

The antibodies can have one or more amino acid substitutions, deletions, insertions, and/or additions. In certain embodiments, the antibodies comprise one of the above-mentioned heavy chain variable domains and one of the above-mentioned light chain variable domains. In certain embodiments, the PD-L1 antibodies or binding fragments thereof comprise one or more CDRs or one or more variable domains with an amino acid sequence at least 85% at least 90%, at least 95%, at least 97%, at least 98%, or at least 99%, identical to the CDR and variable domain sequences set forth in Table 1.

“Identity” refers to the number or percentage of identical positions shared by two amino acid or nucleic acid sequences, taking into account the number of gaps, and the length of each gap, which need to be introduced for optimal alignment of the two sequences. “Substantially identical” means an amino acid sequence which differs only by conservative amino acid substitutions, for example, substitution of one amino acid for another of the same class (e.g., valine for glycine, arginine for lysine, etc.) or by one or more non-conservative substitutions, deletions, or insertions located at positions of the amino acid sequence which do not destroy the function of the protein. Amino acid substitutions can be made, in some cases, by selecting substitutions that do not differ significantly in their effect on maintaining (a) the structure of the peptide backbone in the area of the substitution, (b) the charge or hydrophobicity of the molecule at the target sit; or (c) the bulk of the side chain. For example, naturally occurring residues can be divided into groups based on side-chain properties; (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Examples of substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

Preferably, the amino acid sequence is at least 80%, or at least 85%, or at least 90%, or at least 95% identical to an amino acid sequence disclosed herein. Methods and computer programs for determining sequence similarity are publically available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Antibodies of the invention also include those for which binding characteristics have been improved by direct mutation, methods of affinity maturation, phage display, or chain shuffling. Affinity and specificity may be modified or improved by mutating CDRs and screening for antigen binding sites having the desired characteristics. CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (see, e.g., Hawkins et al., J. Mol. Biol., 226: 889-896 (1992)). For example, phage display vectors containing heavy and light chain variable region genes may be propagated in mutator strains of E. coli (see, e.g., Low et al., J. Mol. Biol., 250: 359-368 (1996)). These methods of mutagenesis are illustrative of the many methods known to one of skill in the art.

To minimize the immunogenicity, antibodies which comprise human constant domain sequences are preferred. The antibodies may be or may combine members of any immunoglobulin class, such as IgG, IgM, IgA, IgD, or IgE, and the subclasses thereof. The antibody class may be selected to optimize effector functions (e.g., complement dependent cytotoxicity (CDC) and antibody dependent cellular cytotoxicity (ADCC)) of natural antibodies.

Certain embodiments of the invention involve the use of PD-L1-binding antibody fragments. An Fv is the smallest fragment that contains a complete heavy and light chain variable domain, including all six hypervariable loops (CDRs). Lacking constant domains, the variable domains are noncovalently associated. The heavy and light chains may be connected into a single polypeptide chain (a “single-chain Fv” or “scFv”) using a linker that allows the V_{H} and V_{L} domains to associate to form an antigen binding site. In an embodiment of the invention, the linker is (Gly-Gly-Gly-Gly-Ser)₃. Since scFv fragments lack the constant domains of whole antibodies, they are considerably smaller than whole antibodies. scFv fragments are also free of normal heavy-chain constant domain interactions with other biological molecules which may be undesired in certain embodiments.

Fragments of an antibody containing V_{H}, V_{L}, and optionally C_{L}, C_{H}1, or other constant domains can also be used. Monovalent fragments of antibodies generated by papain digestion are referred to as Fab and lack the heavy chain hinge region. Fragments generated by pepsin digestion, referred to as F(ab′)₂, retain the heavy chain hinge and are divalent. Such fragments may also be recombinantly produced. Many other useful antigen-binding antibody fragments are known in the art, and include, without limitation, diabodies, triabodies, single domain antibodies, and other monovalent and multivalent forms.

The invention further provides multivalent antigen-binding proteins, which can be in the form, without limitation, of antibodies, antigen-binding fragments thereof, and proteins comprising all or part of antigen-binding portions of antibodies. Multivalent antigen-binding proteins may be monospecific, bispecific, or multispecific. The term specificity refers to the number of different types of antigenic determinants to which a particular molecule can bind. If an immunoglobulin molecule binds to only one type of antigenic determinant, the immunoglobulin molecule is monospecific. If the immunoglobulin molecule binds to different types of antigenic determinants then the immunoglobulin molecule is multispecific.

In an embodiment of the invention, the PD-L1 binding protein has an on rate constant (Kon) of at least about 10²M⁻¹s⁻¹; at least about 10³M⁻¹s⁻¹; at least about 10⁴M⁻¹s⁻¹; at least about 10⁵M⁻¹s⁻¹; or at least about 10⁶M⁻¹s⁻¹, as measured by surface plasmon resonance. In an embodiment, the PD-L1 binding protein has an on rate constant (Kon) between 10²M⁻¹s⁻¹ and 10³M⁻¹s⁻¹; between 10³M⁻¹s⁻¹ and 10⁴M⁻¹s⁻¹; between 10⁴M⁻¹s⁻¹ and 10⁵M⁻¹s⁻¹; or between 10⁵M⁻¹s⁻¹ and 10⁶M⁻¹s⁻¹, as measured by surface plasmon resonance.

In another embodiment the PD-L1 binding protein has an off rate constant (Koff) of at most about 10⁻³s⁻¹; at most about 10⁻⁴s⁻¹; at most about 10⁻⁵s⁻¹; or at most about 10⁻⁶s⁻¹, as measured by surface plasmon resonance. In an embodiment, the PD-L1 binding protein has an off rate constant (Koff) of 10⁻³s⁻¹ to 10⁻⁴s⁻¹; of 10⁻⁴s⁻¹ to 10⁻⁵s⁻¹; or of 10⁻⁵s⁻¹ to 10⁻⁶s⁻¹, as measured by surface plasmon resonance.

In another embodiment the PD-L1 binding protein has a dissociation constant (K_{D}) of at most about 10⁻⁷M; at most about 10⁻⁸M; at most about 10⁻⁹M; at most about 10⁻¹M; at most about 10⁻¹¹M; at most about 10⁻¹²M; or at most 10⁻¹³M. In an embodiment, the binding protein has a dissociation constant (K_{D}) to its targets of 10⁻⁷M to 10⁻⁸M; of 10⁻⁸M to 10⁻⁹M; of 10⁻⁹M to 10⁻¹⁰ M of 10⁻¹⁰M to 10⁻¹¹M; of 10⁻¹¹M to 10⁻¹²M; or of 10⁻¹²M to 10⁻¹³M.

The binding protein described herein may be a conjugate further comprising an imaging agent, a therapeutic agent, or a cytotoxic agent. In an embodiment, the imaging agent is a radiolabel, an enzyme, a fluorescent label, a luminescent label, a bioluminescent label, a magnetic label, or biotin. In another embodiment, the radiolabel is: ³H, ¹⁴C, ³⁵S, ⁹⁰Y, ⁹⁹Tc, ¹¹¹In, ¹²⁵I, ¹³¹I, ¹⁷⁷Lu, or ¹⁵³Sm. In yet another embodiment, the therapeutic or cytotoxic agent is an anti-metabolite, an alkylating agent, an antibiotic, a growth factor, a cytokine, an anti-angiogenic agent, an anti-mitotic agent, an anthracycline, toxin, or an apoptotic agent. As discussed below, immunostimulatory cytokines are of particular importance.

The invention also provides molecules that bind PD-L1 to inhibit immunosuppression, which also promote immune responses by interaction with other ligands or receptors. As exemplified herein, such a molecule combines the PD-L1-binding domain of an antibody with a domain that stimulates NK or T cell function. Such a stimulatory domain can be, without limitation, one that binds to and stimulates a receptor that is responsive to an interleukin or an interferon, such as, without limitation, IL2, IL7, IL15, and IL21. The stimulatory domain exemplified herein is a hybrid domain comprising the sushi domain of the IL15R alpha chain attached to IL15 by a linker (e.g., SEQ ID NO:261). An example of a complete molecule is set forth by SEQ ID NO:262. A nearly identical molecule, modified with two amino acid substitutions in the region between the antibody domain and the IL15R-stimulating domain, to inhibit proteolysis in the region, is set forth by SEQ ID NO:263. As demonstrated herein, a molecule which comprises a PD-L1 binding domain that inhibits immunosuppression, and a second domain which promotes an immune response, provides for increased immune cell activity, compared to two distinct molecules that providing the functions separately.

As exemplified herein, the PD-L1-binding portion of the molecule is an antigen-binding domain of an antibody. Several novel antibody heavy and light chain variable domains and antibodies that include them are provided. According to the invention, the PD-L1-binding portion can be any agent that binds to PD-L1 and blocks immunosuppression. These include anti-PD-L1 antibodies and fragments, not limited to those novel antibodies disclosed herein, as well as peptides and proteins derived from PD1, the natural ligand of PD-L1.

As disclosed herein, the PD-L1-binding domain is linked to a domain that stimulates NK and T cell activity. The domain comprises IL15, and joined to it by a flexible linker, the “sushi” domain from the alpha chain of the IL15 receptor. The sushi domain binds to IL15 with high affinity and the complex of IL15 with the sushi domain is particularly active for stimulating NK and T cell proliferation. What is especially notable is that, as shown in the Examples, treatment with an agent combining the PD-L1-binding domain in the same molecule as the IL15 stimulatory domain is more effective than combined treatment using the PD-L1-binding domain and IL15 stimulatory domain as separate molecules.

Thus, in certain embodiments, the invention contemplates hybrid molecules comprising a domain that bind to PD-L1 and blocks binding to PD1, and a domain that stimulates IL15R, thus proliferation of immune cells. As exemplified, the IL15R stimulatory domain comprises the sushi domain of the IL15R alpha chain joined to IL15 by a flexible linker similar to those employed for, e.g., single chain Fv molecules (i.e., containing 15-20 amino acids which are predominantly serine and glycine. In practice, there are other methods that can be used, which may be preferred for example for manufacturing procedures. Further, one recognizes the domain structures, thus the modular aspects and other features of the disclosed hybrid proteins. For example, the linker joining the sushi domain to IL15 is useful for expressing the hybrid as one polypeptide, but could just as well be replaced by other agents, linkers, or cross linkers. Alternatively, the high affinity of IL15 for the sushi-containing portion of the IL15R alpha chain indicates that the sushi domain and IL15 would form a stable complex that need not be covalent. Similarly, while the exemplified protein comprises an entire antibody constant region, other antigen binding fragments of a PD-L1-binding antibody would suffice.

Accordingly, the invention provides a PD-L1-binding domain linked to an IL15R stimulatory domain, which IL15R stimulatory domain comprises the sushi domain of the IL15R alpha chain or a variant thereof and IL15 or a variant thereof. In certain embodiments, the variants would be 80%, 85%, 87%, 90%, 91%, 92%, 93%, 94%, or 95% identical to the sequences disclosed herein. In one embodiment, the sushi domain of the IL15R alpha chain, and IL15 form a covalent complex. In another embodiment, the sushi domain of the IL15R alpha chain and IL15 form a non-covalent complex. The PD-L1 binding domain can comprise one, two, three, four, five, or six CDRs, or the heavy and or light chain variable domain of an antibody thereof disclosed herein, of be an antigen-binding fragment thereof, or a variant thereof, such as a variant that is 80%, 85%, 87%, 90%, 91%, 92%, 93%, 94%, or 95% identical, or a PD-L1 antibody known in the art that blocks binding to PD-1 or an antigen binding fragment thereof.

It is understood that the anti-PD-L1 antibodies and hybrid proteins of the invention, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, sucrose, polysorbate, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the antibodies.

In the methods of the present invention, a therapeutically effective amount of an antibody of hybrid protein of the invention is administered to a mammal in need thereof. The term “administering” as used herein means delivering the antibodies and fusion proteins of the present invention to a mammal by any method that may achieve the result sought. They may be administered, for example, intravenously or intramuscularly. Although the exemplified antibodies of the invention are particularly useful for administration to humans, they may be administered to other mammals as well. The term “mammal” as used herein is intended to include, but is not limited to, humans, laboratory animals, domestic pets and farm animals. “Therapeutically effective amount” means an amount of antibody of the present invention that, when administered to a mammal, is effective in producing the desired therapeutic effect, such as inhibiting kinase activity.

Antibodies and hybrid proteins of the invention are useful for inhibiting tumors and other neoplastic diseases, as well as treating other pathologic conditions associated with immunosuppression. Tumors that can be treated include primary tumors, metastatic tumors, and refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents, but recur up to five years, sometimes up to ten years or longer after treatment is discontinued. The antibodies are effective for treating vascularized tumors and tumor that are not vascularized, or not yet substantially vascularized.

Examples of solid tumors which may be accordingly treated include breast carcinoma, lung carcinoma, colorectal carcinoma, pancreatic carcinoma, glioma and lymphoma. Some examples of such tumors include epidermoid tumors, squamous tumors, such as head and neck tumors, colorectal tumors, prostate tumors, breast tumors, lung tumors, including small cell and non-small cell lung tumors, pancreatic tumors, thyroid tumors, ovarian tumors, and liver tumors. Other examples include Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma. Examples of vascularized skin cancers for which the antagonists of this invention are effective include squamous cell carcinoma, basal cell carcinoma and skin cancers that can be treated by suppressing the growth of malignant keratinocytes, such as human malignant keratinocytes.

Examples of non-solid tumors include leukemia, multiple myeloma and lymphoma that are unresponsive to cytokines, such as IL15. Some examples of leukemias include acute myelogenous leukemia (AML), chronic myelogenous leukemia (CML), acute lymphocytic leukemia (ALL), chronic lymphocytic leukemia (CLL), erythrocytic leukemia or monocytic leukemia. Some examples of lymphomas include Hodgkin's and non-Hodgkin's lymphoma.

The PD-L1 antibodies and immune cell stimulating hybrid proteins of the invention are also used in the treatment of viral infections. PD-1 expression on T cells correlates with viral load in HIV and HCV infected patients and PD-1 expression has been identified as a marker for exhausted virus-specific CD8+ T cells. For example, PD-1⁺CD8⁺ T cells show impaired effector functions and PD-1 associated T cell exhaustion which can be restored by blocking the PD-1/PD-L1 interaction. This results in recovery of virus-specific CD8+ T cell mediated immunity, indicating that interrupting PD-1 signaling using an antagonistic antibody restores T-cell effector functions. Immunotherapy based on the blockade of PD-1/PD-L1 results in breakdown of T-cell tolerance not only to tumor antigens, but also provides a strategy to reactivate virus-specific effector T cells and eradicate pathogens in chronic viral infections. Accordingly, the antibodies and hybrid proteins of the invention are useful to treat chronic viral infections, including, without limitation, HCV and HIV, and lymphocytic choriomeningitis virus (LCMV).

The antibodies and hybrid proteins of the invention can be advantageously administered with second agents to patients in need thereof. For example, in some embodiments, an antibody or hybrid protein of the invention is administered to a subject with an anti-neoplastic agent. In some embodiments, an antibody or hybrid protein of the invention is administered to a subject with a second angiogenesis inhibitor. In some embodiments, an antibody or hybrid protein of the invention is administered with an anti-inflammatory agent or an immunosuppressant.

Antineoplastic agents include cytotoxic chemotherapeutic agents, targeted small molecules and biological molecules, and radiation. Non-limiting examples of chemotherapeutic agents include cisplatin, dacarbazine (DTIC), dactinomycin, irinotecan, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, taxol and combinations thereof.

Targeted small molecules and biological molecules include, without limitation, inhibitors of components of signal transduction pathways, such as modulators of tyrosine kinases and inhibitors of receptor tyrosine kinases, and agents that bind to tumor-specific antigens. Non-limiting examples of growth factor receptors involved in tumorigenesis are the receptors for platelet-derived growth factor (PDGFR), insulin-like growth factor (IGFR), nerve growth factor (NGFR), and fibroblast growth factor (FGFR), and receptors of the epidermal growth factor receptor family, including EGFR (erbB1), HER2 (erbB2), erbB3, and erbB4.

EGFR antagonists include antibodies that bind to EGFR or to an EGFR ligand, and inhibits ligand binding and/or receptor activation. For example, the agent can block formation of receptor dimers or heterodimer with other EGFR family members. Ligands for EGFR include, for example, EGF, TGF-α amphiregulin, heparin-binding EGF (HB-EGF) and betaregullulin. An EGFR antagonist can bind externally to the extracellular portion of EGFR, which may or may not inhibit binding of the ligand, or internally to the tyrosine kinase domain. EGFR antagonists further include agents that inhibit EGFR-dependent signal transduction, for example, by inhibiting the function of a component of the EGFR signal transduction pathway. Examples of EGFR antagonists that bind EGFR include, without limitation, biological molecules, such as antibodies (and functional equivalents thereof) specific for EGFR, and small molecules, such as synthetic kinase inhibitors that act directly on the cytoplasmic domain of EGFR.

Small molecule and biological inhibitors include inhibitors of epidermal growth factor receptor (EGFR), including gefitinib, erlotinib, and cetuximab, inhibitors of HER2 (e.g., trastuzumab, trastuzumab emtansine (trastuzumab-DM1; T-DM1) and pertuzumab), anti-VEGF antibodies and fragments (e.g., bevacizumab), antibodies that inhibit CD20 (e.g., rituximab, ibritumomab), anti-VEGFR antibodies (e.g., ramucirumab (IMC-1121B), IMC-1C11, and CDP791), anti-PDGFR antibodies, and imatinib. Small molecule kinase inhibitors can be specific for a particular tyrosine kinase or be inhibitors of two or more kinases. For example, the compound N-(3,4-dichloro-2-fluorophenyl)-7-({[(3aR,6aS)-2-methyloctahydrocyclopenta[c] pyrrol-5-yl]methyl}oxy)-6-(methyloxy)quinazolin-4-amine (also known as XL647, EXEL-7647 and KD-019) is an in vitro inhibitor of several receptor tyrosine kinases (RTKs), including EGFR, EphB4, KDR (VEGFR), Flt4 (VEGFR3) and ErbB2, and is also an inhibitor of the SRC kinase, which is involved in pathways that result in nonresponsiveness of tumors to certain TKIs. In an embodiment of the invention, treatment of a subject in need comprises administration of a rho-kinase inhibitor of Formula I and administration of KD-019.

Dasatinib (BMS-354825; Bristol-Myers Squibb, New York) is another orally bioavailable, ATP-site competitive Src inhibitor. Dasatanib also targets Bcr-Ab1 (FDA-approved for use in patients with chronic myelogenous leukemia (CML) or Philadelphia chromosome positive (Ph+) acute lymphoblastic leukemia (ALL)) as well as c-Kit, PDGFR, c-FMS, EphA2, and SFKs. Two other oral tyrosine kinase inhibitor of Src and Bcr-Ab1 are bosutinib (SKI-606) and saracatinib (AZD0530).

In an embodiment of the invention, a PD-L1 antibody or conjugate of the invention is used in combination with an anti-viral agent to treat a chronic virus infection. For example, tor HCV, the following agents can be used. HCV protease inhibitors include, without limitation, boceprevir, telaprevir (VX-950), ITMN-191, SCH-900518, TMC-435, BI-201335, MK-7009, VX-500, VX-813, BMS790052, BMS650032, and VBY376. HCV nonstructural protein 4B (NS4B) inhibitors include, but are not limited to, clemizole, and other NS4B-RNA binding inhibitors, including but not limited to benzimidazole RBIs (B-RBIs) and indazole RBIs (I-RBIs). HCV nonstructural protein 5A (NS5A) inhibitors include, but are not limited to, BMS-790052, A-689, A-831, EDP239, GS5885, and PP1461. HCV polymerase (NS5B) inhibitors include, but are not limited to nucleoside analogs (e.g., valopicitabine, R1479, R1626, R7128), nucleotide analogs (e.g., IDX184, PSI-7851, PSI-7977, and non-nucleoside analogs (e.g., filibuvir, HCV-796, VCH-759, VCH-916, ANA598, VCH-222 (VX-222), BI-207127, MK-3281, ABT-072, ABT-333, GS9190, BMS791325). Also, ribavirin or a ribavirin analog such as Taribavirin (viramidine; ICN 3142), Mizoribine, Merimepodib (VX-497), Mycophenolate mofetil, and Mycophenolate can be used.

In certain embodiments, a dose of an antibody or hybrid protein of the invention is administered to a subject every day, every other day, every couple of days, every third day, once a week, twice a week, three times a week, or once every two weeks. In other embodiments, two, three or four doses of a compound or a composition is administered to a subject every day, every couple of days, every third day, once a week or once every two weeks. In some embodiments, a dose(s) of a compound or a composition is administered for 2 days, 3 days, 5 days, 7 days, 14 days, or 21 days. In certain embodiments, a dose of a compound or a composition is administered for 1 month, 1.5 months, 2 months, 2.5 months, 3 months, 4 months, 5 months, 6 months or more.

Methods of administration include but are not limited to parenteral, intradermal, intravitrial, intramuscular, intraperitoneal, intravenous, subcutaneous, intranasal, epidural, oral, sublingual, intranasal, intracerebral, intravaginal, transdermal, transmucosal, rectally, by inhalation, or topically, particularly to the ears, nose, eyes, or skin. The mode of administration is left to the discretion of the practitioner. In most instances, administration will result in the release of a compound into the bloodstream. For treatment of ocular disease, intravitrial administration of biological agents is preferred.

In specific embodiments, it may be desirable to administer a compound locally. This may be achieved, for example, and not by way of limitation, by local infusion, topical application, by injection, by means of a catheter, or by means of an implant, said implant being of a porous, non-porous, or gelatinous material, including membranes, such as sialastic membranes, or fibers. In such instances, administration may selectively target a local tissue without substantial release of a compound into the bloodstream.

Pulmonary administration can also be employed, e.g., by use of an inhaler or nebulizer, and formulation with an aerosolizing agent, or via perfusion in a fluorocarbon or synthetic pulmonary surfactant. In certain embodiments, a compound is formulated as a suppository, with traditional binders and vehicles such as triglycerides.

In another embodiment, a compound is delivered in a vesicle, in particular a liposome (See Langer, 1990, Science 249:1527-1533; Treat et al., in Liposomes in the Therapy of Infectious Disease and Bacterial infection, Lopez-Berestein and Fidler (eds.), Liss, New York, pp. 353-365 (1989); Lopez Berestein, ibid., pp. 317-327; see generally ibid.).

In another embodiment, a compound is delivered in a controlled release system (See, e.g., Goodson, in Medical Applications of Controlled Release, supra, vol. 2, pp. 115-138 (1984)). Examples of controlled-release systems are discussed in the review by Langer, 1990, Science 249:1527-1533 may be used. In one embodiment, a pump may be used (See Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (See Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; See also Levy et al., 1985, Science 228:190; During et al., 1989, Ann. Neurol. 25:351; Howard et al., 1989, J. Neurosurg. 71:105).

The above-described administration schedules are provided for illustrative purposes only and should not be considered limiting. A person of ordinary skill in the art will readily understand that all doses are within the scope of the invention.

It is to be understood and expected that variations in the principles of invention herein disclosed may be made by one skilled in the art and it is intended that such modifications are to be included within the scope of the present invention.

Throughout this application, various publications are referenced. These publications are hereby incorporated into this application by reference in their entireties to more fully describe the state of the art to which this invention pertains. The following examples further illustrate the invention, but should not be construed to limit the scope of the invention in any way.

### EXAMPLES

### Mixed-Lymphocyte Reactions:

CD14 positive monocytes were isolated by negative selection from whole blood using RosetteSep human monocyte enrichment kit (StemCell technologies). Immature monocyte-derived dendritic cells (mo-DC) were generated by culturing CD14 positive cells in IMDM supplemented with 10% FBS with 150 ng/mL GM-CSF and 50 ng/mL IL-4 for 6 to 7 days. CD4 positive cells were negatively isolated from whole blood using RosetteSep human CD4 enrichment kit (StemCell technologies). Mo-DC and CD4 positive cells from a different donor were then co-cultured at a ratio 1 to 10 of mo-DC to CD4 cells respectively. To assess blocking function of anti-PDL1 antibodies, increasing amount of anti-PDL1 antibodies was added in the beginning of co-culture. In some cases, increasing amount of IL15 was also added at the beginning of co-culture. At day 6 or 7, the supernatants were collected for measurements of secreted IL-2 and IFN□□by ELISA. The number of CD4 cells and expression of the proliferation marker, Ki67, were evaluated by flow cytometry.

### Activation of PBMC:

PBMC was isolated from whole blood using Histopaque-1077 (Sigma), cultured in IMDM supplemented with 10% FBS and activated by either SEB (0.1 ug/mL), PHA (1 ug/mL) or anti-CD3 clone HiT3a (1 ug/mL, eBioscience) for 3 to 7 days. Binding of either anti-PDL1 antibodies or anti-PDL1-SD15 fusion proteins were evaluated in activated PBMC after 3 days by flow cytometry. Functional assessment of anti-PDL1 antibodies were done by addition of increasing amount of anti-PDL1 antibodies during PBMC activation with SEB. At day 2 or 3 supernatants were collected for measurements of IL-2 and IFN□. In the case of anti-PDL1-SD15 fusion proteins, PBMC were cultured in the presence of either anti-PDL1-SD15 or anti-PDL1 antibodies, with no other activations. At day 6, cells were collected, and the numbers of CD8 and granzymeB, CD8 and perforin, and CD4 cells were evaluated by flow cytometry.

### Activation of CD4 and CD8 Cells:

CD4 and CD8 positive cells were negatively isolated from whole blood using RosetteSep enrichment kits (StemCell technologies). CD4 cells were activated by either anti-CD3 or anti-CD3 and PDL1Fc coated beads in IMDM, 10%FBS in the presence of anti-PDL1 antibodies. At day 5, supernatants were collected for IFN□ measurements by ELISA, and cells were evaluated for expression of the proliferation marker Ki67 using flow cytometry. CD8 cells were activated by anti-CD3 coated beads and either IL15 or anti-PDL1-SD15 fusion proteins. In some cases anti-CD3 and PDL1Fc was used in place of anti-CD3 coated beads. At day 6 or 7 the supernatants were collected for measurements of IFN□ and TNF□ secretions by ELISA. The cells were collected for measurements of CD8 activation by granzymeB and perforin markers using flow cytometry.

### Nomenclature of Antibody-Fusion Proteins:

In experiments with anti-PD-L1 IL15 fusion proteins, shorter names for the fusion proteins are identified in the legends. The fusion protein tcc1D7HC-SD15 is identified in figure legends as cD7-SD15. The fusion protein tcc1F8HC-SD15 is identified in figure legends as F8-SD15.

Specific High Affinity Antibodies to PD-L1 from Phage-Display Library

Anti-PD-L1 antibodies with high affinity were obtained using a phage display library. In one procedure, phage Fabs amplified from Dyax libraries were panned on either recombinant human PDL1-Fc (PDL1 ECD and human Fc fusion protein, Q9NZQ7) or murine PDL1-Fc (Q9EP73) which were immobilized on immune-tubes for three rounds. The ELISA positive clones from round (R2) and round 3 (R3) were sequenced.

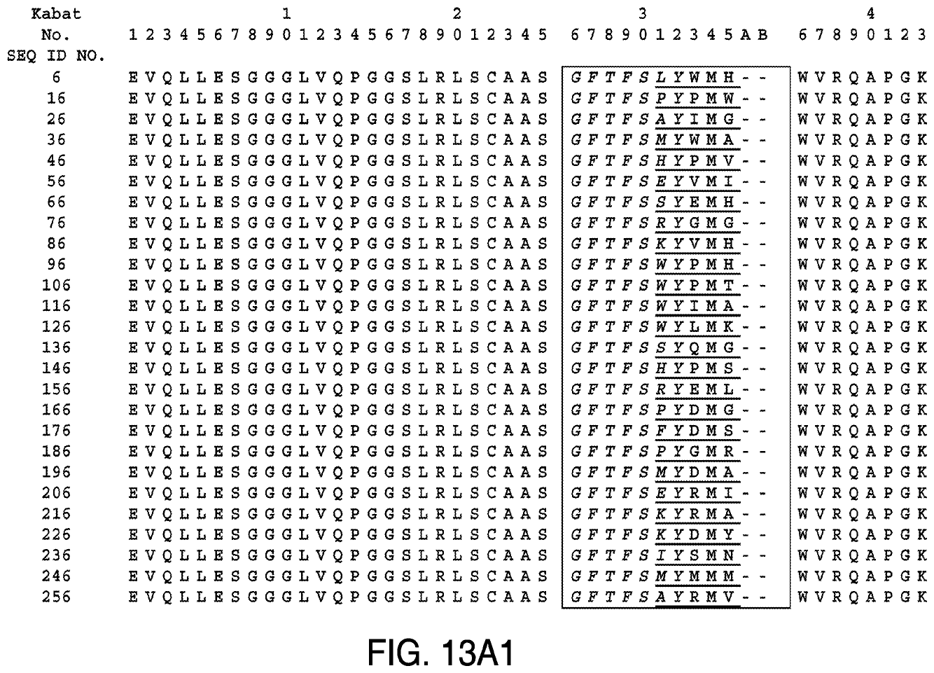

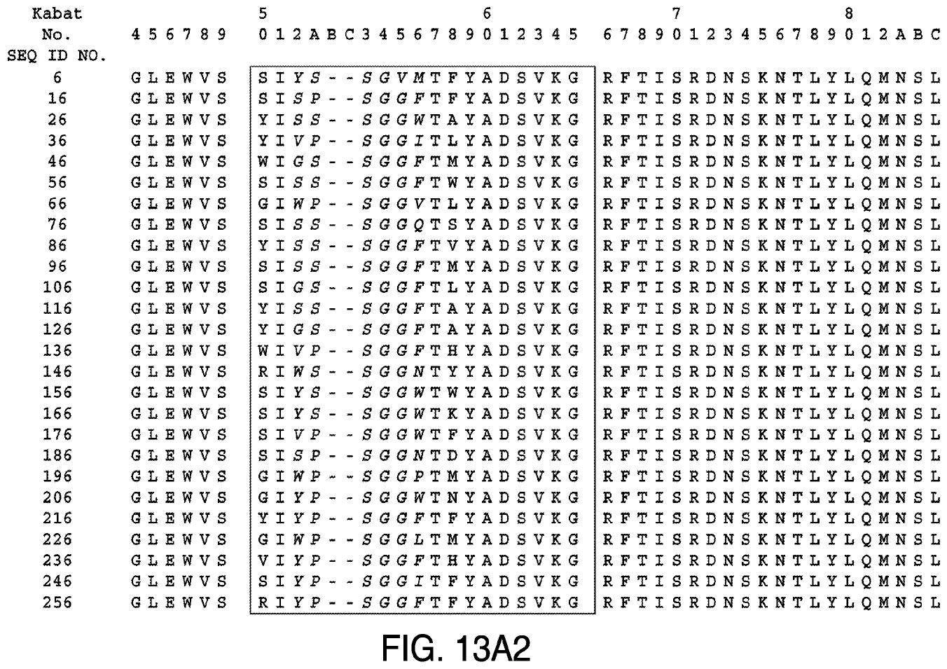

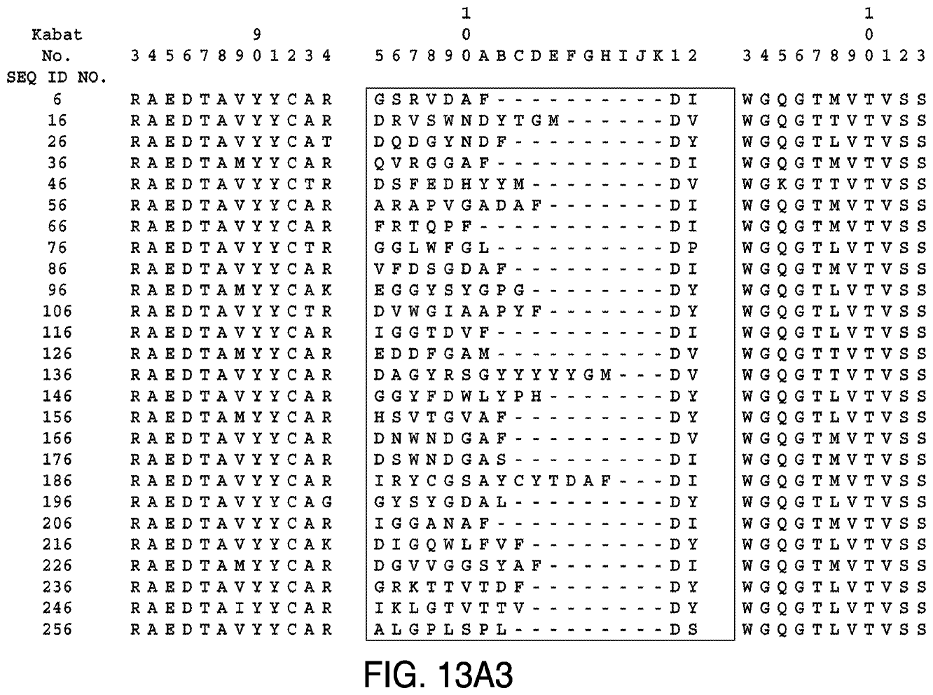

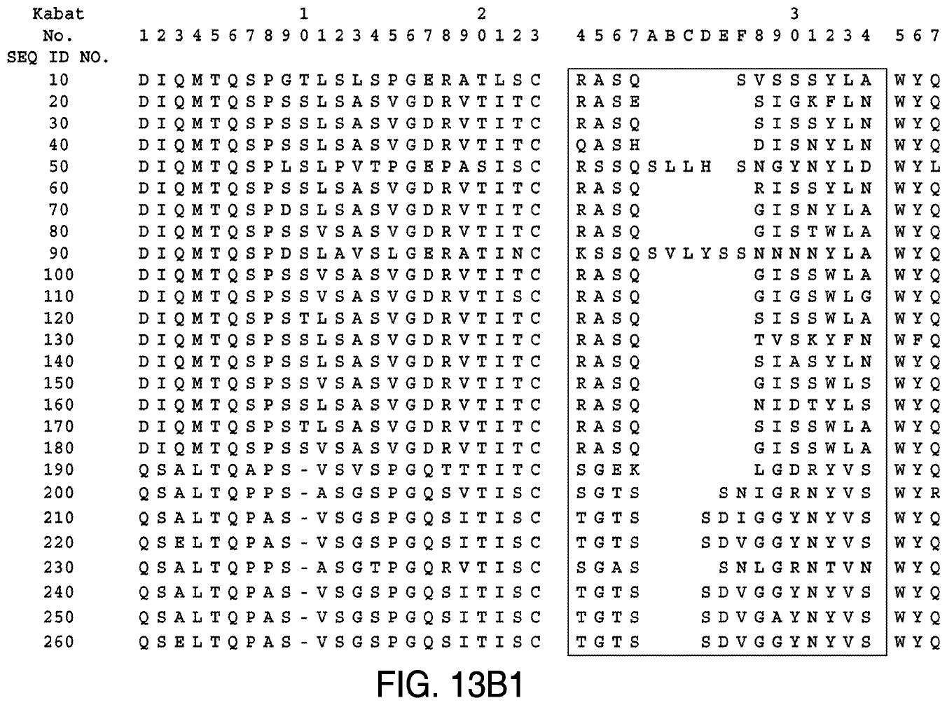

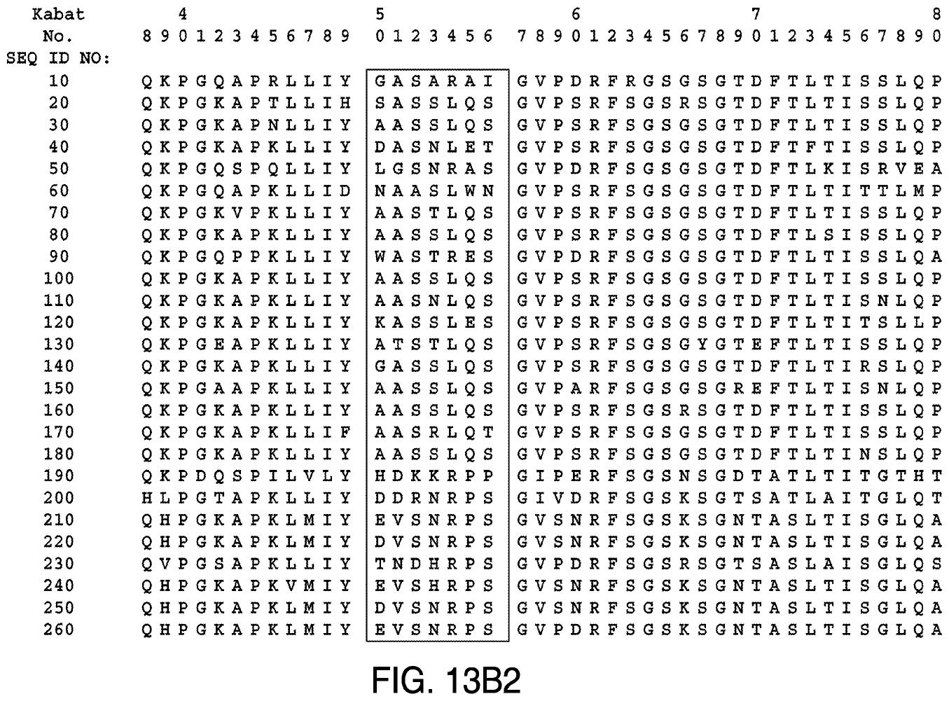

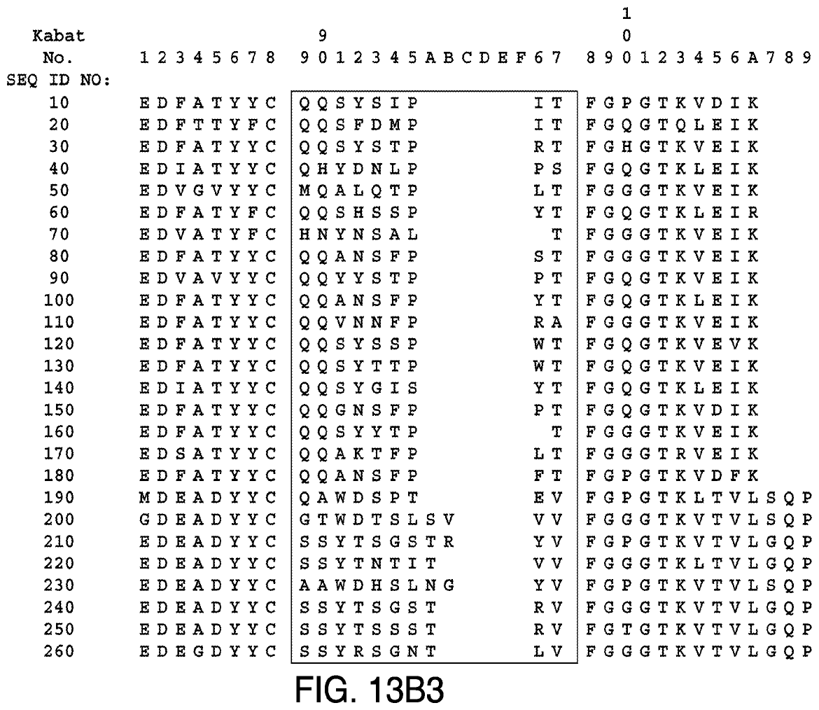

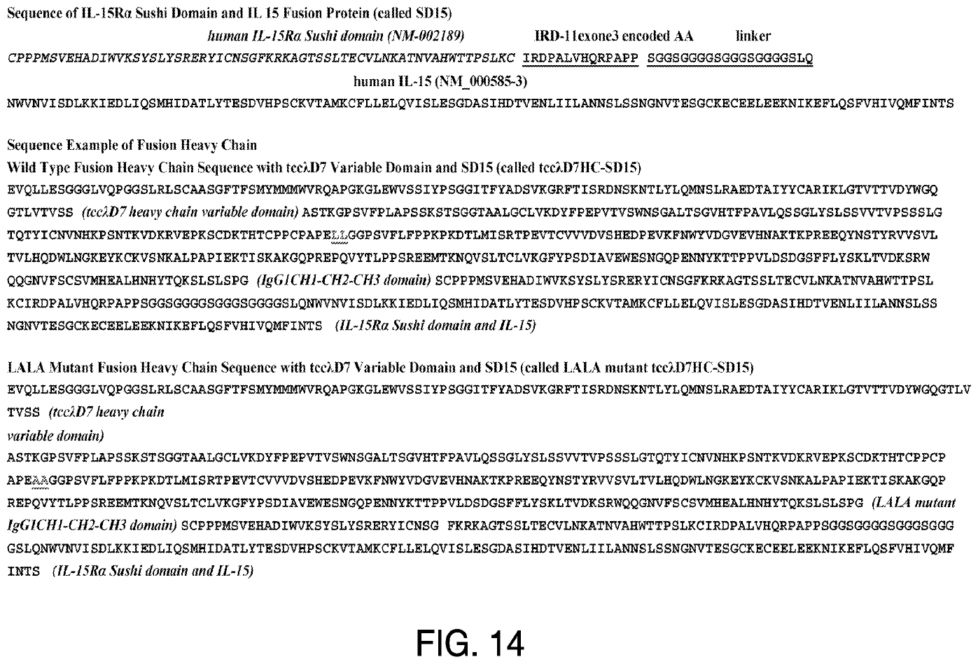

In a second procedure, phage Fabs amplified from the Dyax libraries were panned on recombinant human PDL1-Fc (PDL1 ECD and human Fc fusion protein, Q9NZQ7) for the first round, and then panned on activated T cells for second round. For third round, either the activated T cells or recombinant human PDL1-Fc were used for the panning. Clones which can bind to both soluble PDL1-Fc and cell expressed PDL1-Fc were sequenced. V_{H} and V_{L} variable domain sequences of these antibodies are set forth in FIGS. 13A1, 13A2, 13A3, 13B1, 13B2, 13B3 and the rows 1-26 of Table 1.

Unique clones were converted to IgG for the further characterization. The variable domains were inserted to Dyax expression vector pBhl. Both wild type CH1-CH2-CH3 domains and mutated CH1-CH2-CH3 (L234A and L235A, also referred to herein as LALA mutants) were prepared in the IgG format.

TABLE 1
Antibody Amino Acid Sequences by SEQ ID NO.

| | V_{H} CDRs | | | | | | | | | |
|---|---|---|---|---|---|---|---|---|---|---|
| | H1 | H1 | H2 | H2 | | | V_{L} CDRs | | | |
| Mab | (K) | (C) | (K) | (C) | H3 | V_{H} | L1 | L1 | L3 | V_{L} |
| R2κA3 | 1 | 2 | 3 | 4 | 5 | 6 | 7 | 8 | 9 | 10 |
| R2κA4 | 11 | 12 | 13 | 14 | 15 | 16 | 17 | 18 | 19 | 20 |
| R2κA6 | 21 | 22 | 23 | 24 | 25 | 26 | 27 | 28 | 29 | 30 |
| R2κF4 | 31 | 32 | 33 | 34 | 35 | 36 | 37 | 38 | 39 | 40 |
| R2κH5 | 41 | 42 | 43 | 44 | 45 | 46 | 47 | 48 | 49 | 50 |
| R2κH6 | 51 | 52 | 54 | 54 | 55 | 56 | 57 | 58 | 59 | 60 |
| R2κH3 | 61 | 62 | 63 | 64 | 65 | 66 | 67 | 68 | 69 | 70 |
| sR3κA8 | 71 | 72 | 73 | 74 | 75 | 76 | 77 | 78 | 79 | 80 |
| sR3κA9 | 81 | 82 | 83 | 84 | 85 | 86 | 87 | 88 | 89 | 90 |
| sR3κB2 | 91 | 92 | 93 | 94 | 95 | 96 | 97 | 98 | 99 | 100 |
| sR3κB5 | 101 | 102 | 103 | 104 | 105 | 106 | 107 | 108 | 109 | 110 |
| tccR3κA8 | 111 | 112 | 113 | 114 | 115 | 116 | 117 | 118 | 119 | 120 |
| tccR3κA11 | 121 | 122 | 123 | 124 | 125 | 126 | 127 | 128 | 129 | 130 |
| tccR3κB7 | 131 | 132 | 133 | 134 | 135 | 136 | 137 | 138 | 139 | 140 |
| tccR3κD9 | 141 | 142 | 143 | 144 | 145 | 146 | 147 | 148 | 149 | 150 |
| tccκF10 | 161 | 162 | 163 | 164 | 165 | 166 | 157 | 158 | 159 | 160 |
| tctR3κA4 | 161 | 162 | 163 | 164 | 165 | 166 | 167 | 168 | 169 | 170 |
| tctR3κF8 | 171 | 172 | 173 | 174 | 175 | 176 | 177 | 178 | 179 | 180 |
| R2λA7 | 181 | 182 | 183 | 184 | 185 | 186 | 187 | 188 | 189 | 190 |
| R2λB12 | 191 | 192 | 193 | 194 | 195 | 196 | 197 | 198 | 199 | 200 |
| R2λD12 | 201 | 202 | 203 | 204 | 205 | 206 | 207 | 208 | 209 | 210 |
| sR3λD7 | 211 | 212 | 213 | 214 | 215 | 216 | 217 | 218 | 219 | 220 |
| sR3λE1 | 221 | 222 | 223 | 224 | 225 | 226 | 227 | 228 | 229 | 230 |
| tccλF8 | 231 | 232 | 233 | 234 | 235 | 236 | 237 | 238 | 239 | 240 |
| tccλD7 | 241 | 242 | 243 | 244 | 245 | 246 | 247 | 248 | 249 | 250 |
| tctR3λH4 | 251 | 252 | 253 | 254 | 255 | 256 | 257 | 258 | 259 | 260 |
| #101 | 264 | — | 243 | 244 | 245 | 265 | 247 | 248 | 249 | 250 |
| #102 | 266 | — | 243 | 244 | 245 | 267 | 247 | 248 | 249 | 250 |
| #103 | 268 | — | 243 | 244 | 245 | 269 | 247 | 248 | 249 | 250 |
| #104 | 270 | — | 243 | 244 | 245 | 271 | 247 | 248 | 249 | 250 |
| #105 | 272 | — | 243 | 244 | 245 | 273 | 247 | 248 | 249 | 250 |
| #106 | 274 | — | 243 | 244 | 245 | 275 | 247 | 248 | 249 | 250 |
| #107 | 276 | — | 243 | 244 | 245 | 277 | 247 | 248 | 249 | 250 |
| #108 | 278 | — | 243 | 244 | 245 | 279 | 247 | 248 | 249 | 250 |
| #109 | 280 | — | 243 | 244 | 245 | 281 | 247 | 248 | 249 | 250 |
| #110 | 282 | — | 243 | 244 | 245 | 283 | 247 | 248 | 249 | 250 |
| #111 | 284 | — | 243 | 244 | 245 | 285 | 247 | 248 | 249 | 250 |
| #112 | 286 | — | 243 | 244 | 245 | 287 | 247 | 248 | 249 | 250 |
| #113 | 288 | — | 243 | 244 | 245 | 289 | 247 | 248 | 249 | 250 |
| #114 | 290 | — | 243 | 244 | 245 | 291 | 247 | 248 | 249 | 250 |
| #115 | 292 | — | 243 | 244 | 245 | 293 | 247 | 248 | 249 | 250 |
| #116 | 294 | — | 243 | 244 | 245 | 295 | 247 | 248 | 249 | 250 |
| #117 | 296 | — | 243 | 244 | 245 | 297 | 247 | 248 | 249 | 250 |
| #118 | 298 | — | 243 | 244 | 245 | 299 | 247 | 248 | 249 | 250 |
| #119 | 300 | — | 243 | 244 | 245 | 301 | 247 | 248 | 249 | 250 |
| #120 | 302 | — | 243 | 244 | 245 | 303 | 247 | 248 | 249 | 250 |
| #121 | 304 | — | 243 | 244 | 245 | 305 | 247 | 248 | 249 | 250 |
| #122 | 306 | — | 243 | 244 | 245 | 307 | 247 | 248 | 249 | 250 |
| #123 | 308 | — | 243 | 244 | 245 | 309 | 247 | 248 | 249 | 250 |
| #124 | 310 | — | 243 | 244 | 245 | 311 | 247 | 248 | 249 | 250 |
| #125 | 312 | — | 243 | 244 | 245 | 313 | 247 | 248 | 249 | 250 |

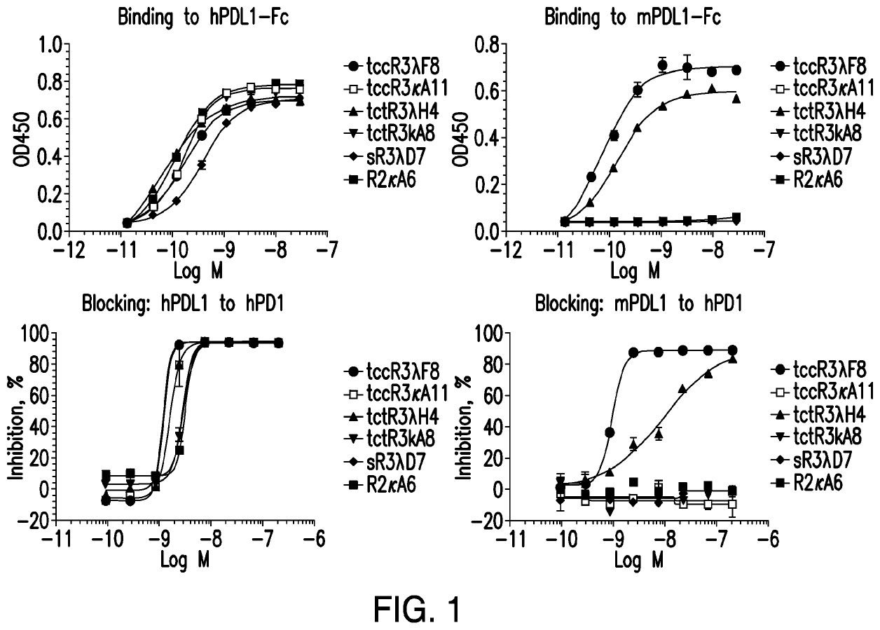

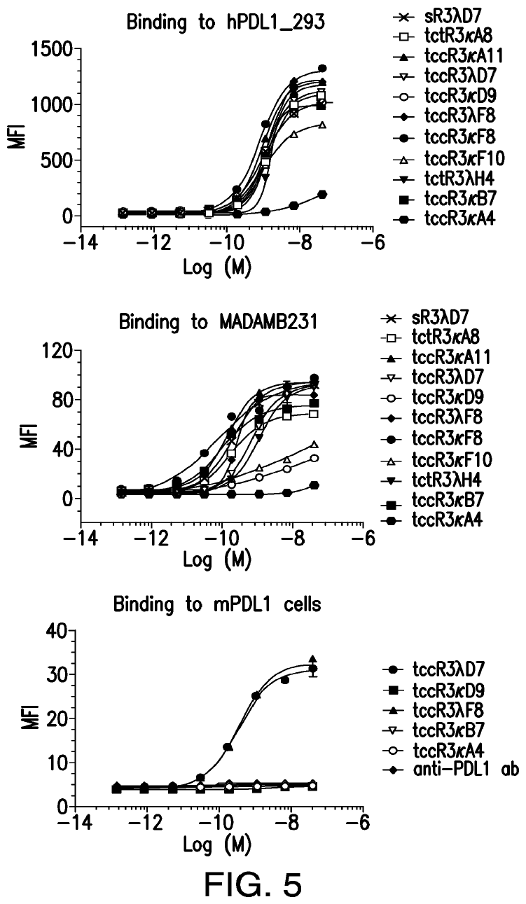

These antibodies were verified to have specific binding to PD-L1 by solid-phase ELISA (FIGS. 1-4) and HEK-293 cells (FIG. 5). Blocking of PD-1:PD-L1 interactions in the presence of these antibodies was determined by solid phase ELISA and by 293-HEK cells expressing PD-L1. Biacore was used to calculate the affinity constant for each antibody.

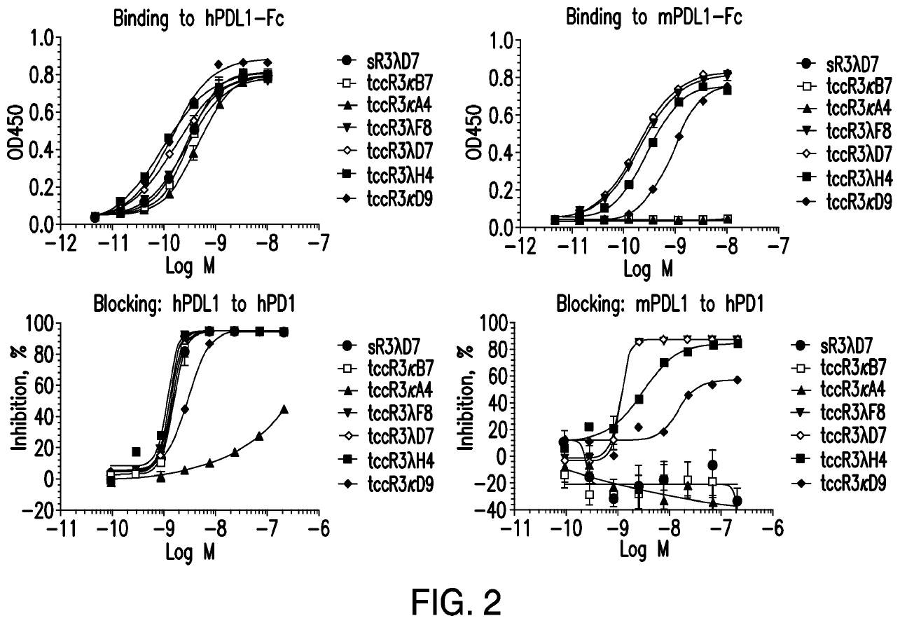

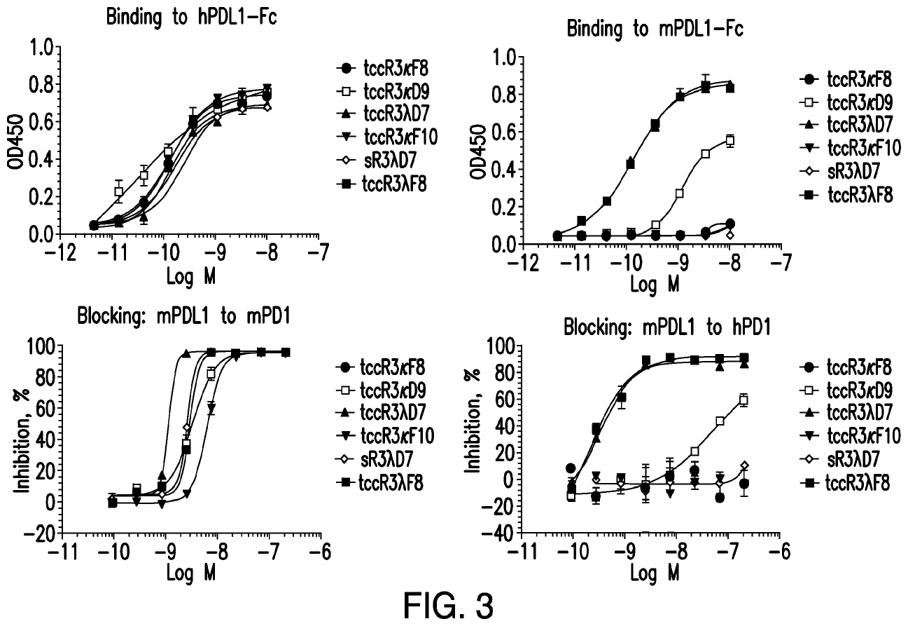

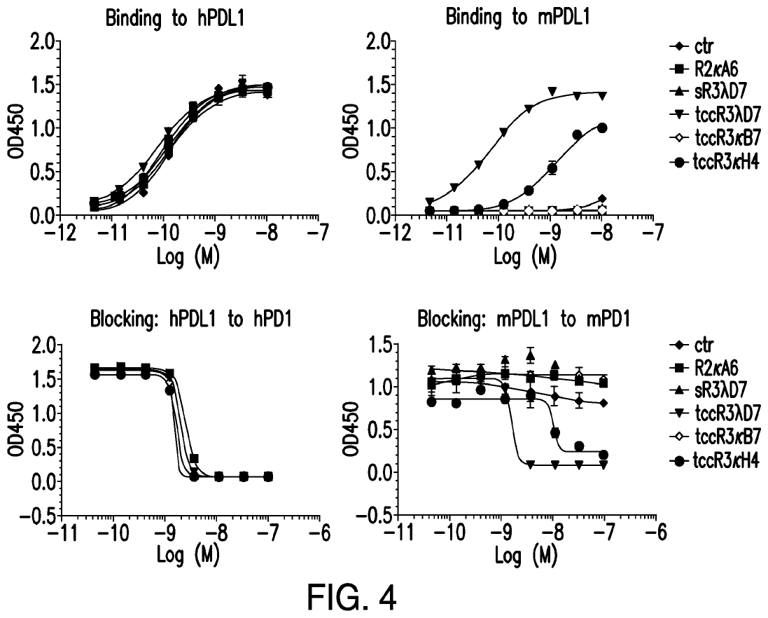

TABLE 2
EC₅₀ and IC₅₀ for antibodies of FIGS. 1-4

| FIG. 1 | tccR3λF8 | tccR3κA11 | tccR3λH4 | tctR3κA8 | sR3λD7 | R2κA6 |
|---|---|---|---|---|---|---|
| h_EC₅₀ | 0.167 | 0.172 | 0.056 | 0.106 | 0.388 | 0.117 |
| h_IC₅₀ | 1.19 | 1.58 | 1.17 | 2.94 | 2.89 | 3.17 |
| m_EC₅₀ | 0.0714 | ND | 0.144 | ND | ND | ND |
| m_IC₅₀ | 0.925 | ND | 9.14 | ND | ND | ND |

| FIG. 2 | sR3λD7 | tccR3κB7 | tccR3κA4 | tccR3λF8 | tccR3λD7 | tccR3λH4 | tccR3κD9 |
|---|---|---|---|---|---|---|---|
| h_EC₅₀ | 0.255 | 0.3 | 0.443 | 0.265 | 0.155 | 0.0947 | 0.132 |
| h_IC₅₀ | 1.47 | 1.26 | ND | 1.36 | 1.24 | 1.12 | 2.75 |
| m_EC₅₀ | ND | ND | ND | 0.205 | 0.185 | 0.282 | ND |
| m_IC₅₀ | ND | ND | ND | 1.08 | 1.13 | 2.88 | ND |

| FIG. 3 | tccR3κF8 | tccR3κD9 | tccR3λD7 | tccR3κF10 | sR3λD7 | tccR3F8 |
|---|---|---|---|---|---|---|
| h_EC₅₀ | 0.13 | 0.0259 | 0.14 | 0.137 | 0.244 | 0.174 |
| h_IC₅₀ | 2.78 | 3.23 | 1.09 | 6.22 | 2.52 | 2.89 |
| m_EC₅₀ | ND | 1.31 | 0.13 | ND | ND | 0.13 |
| m_IC₅₀ | ND | 52.3 | 0.349 | ND | ND | 0.184 |

| FIG. 4 | control | R2κA6 | sR3λD7 | tccR3λD7 | tccR3κB7 | tccR3λH4 |
|---|---|---|---|---|---|---|
| h_EC₅₀ | 0.146 | 0.0909 | 0.129 | 0.0682 | 0.126 | 0.117 |
| h_IC₅₀ | 1.49 | 2 | 2.55 | 1.97 | 2.05 | 1.68 |
| m_EC₅₀ | ND | ND | ND | 0.0622 | ND | ND |
| m_IC₅₀ | ND | ND | ND | 1.58 | ND | ND |

TABLE 3
EC₅₀ on PDLI-293, MDA-MB-231, and mPDLI cells

| | PDLI-293 | MDA-MB-231 | mPDL1 |
|---|---|---|---|
| sR3λD7 | 1.377 × 10⁻⁹ | 2.138 × 10⁻¹⁰ | ND |
| tctR3κA8 | 1.179 × 10⁻⁹ | 1.886 × 10⁻¹⁰ | ND |
| tccR3κA11 | 8.731 × 10⁻¹⁰ | 1.437 × 10⁻¹⁰ | ND |
| tccR3λD7 | 1.153 × 10⁻⁹ | 6.943 × 10⁻¹⁰ | 3.413 × 10⁻¹⁰ |
| tccR3κD9 | 7.886 × 10⁻¹⁰ | 1.241 × 10⁻⁸ | 4.004 × 10⁻⁹ |
| tccR3λF8 | 1.335 × 10⁻⁹ | 2.610 × 10⁻¹⁰ | 3.695 × 10⁻¹⁰ |
| tccR3κF8 | 7.430 × 10⁻¹⁰ | 7.777 × 10⁻¹¹ | ND |
| tccR3κF10 | 9.143 × 10⁻¹⁰ | 1.922 × 10⁻⁸ | ND |
| tccR3λH4 | 1.410 × 10⁻⁹ | 1.049 × 10⁻⁹ | ND |
| tccR3κB7 | 9.732 × 10⁻¹⁰ | 9.833 × 10⁻¹¹ | 2.688 × 10⁻⁹ |
| tccR3κA4 | 9.062 × 10⁻⁸ | 0.0001903 | 2.025 × 10⁻¹⁰ |

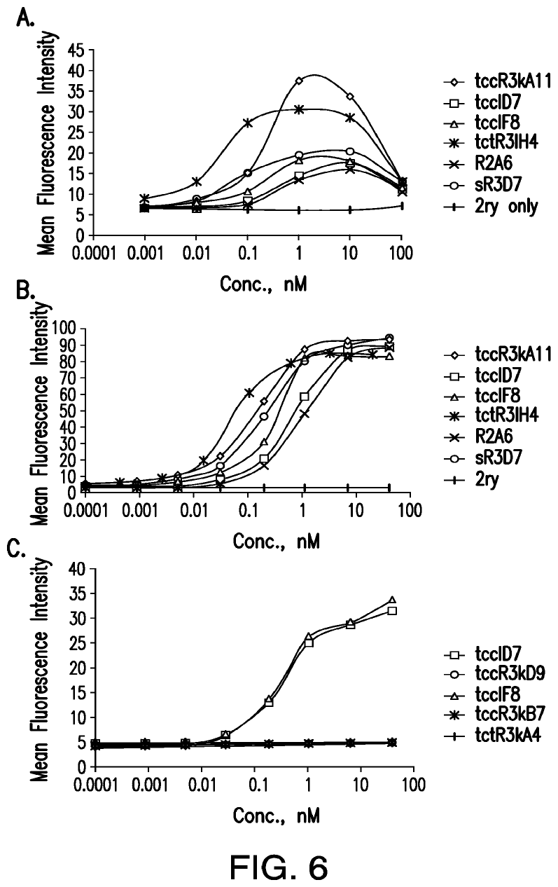

These antibodies were also verified for binding on native cells expressing PD-L1 by binding to immature monocyte-derived dendritic cells (FIG. 6A), the human PD-L1-expressing breast cancer line MDA-MB-231 cells (FIG. 6B), the mouse PD-L1-expressing tumor line B16-F10 cells (FIG. 6C) as well as human activated CD4 and CD8 T cells.

### Functionally Active Anti-PD-L1 Antibodies Block PD-1 PD-L1 Interaction and Increase T Cell Proliferation and Activation

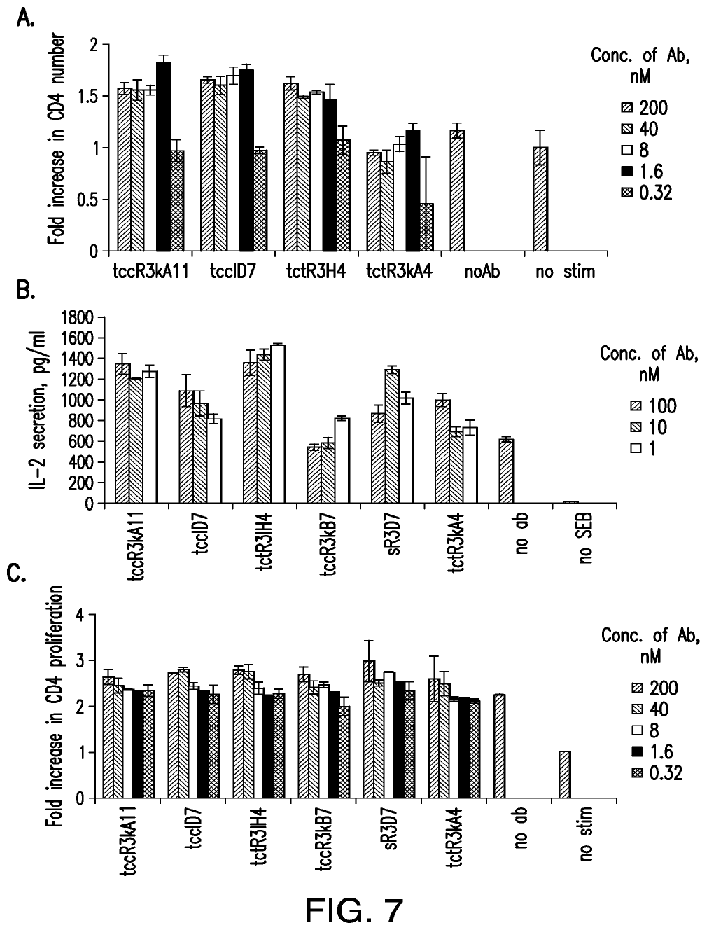

High affinity binding anti-PD-L1 antibodies were evaluated for their function to block PD-1 PD-L1 interactions and increase T cell proliferation. Negatively purified CD4 T cells were activated in vitro with either □CD3 or □CD3 and PD-L1Fc coated beads in the presence of anti-PD-L1 antibodies. CD4 cells stimulated with □CD3 and PD-L1Fc coated beads showed lower proliferation and IFN□ as well as IL-2 secretions as compared to CD4 cells stimulated with □CD3 only coated beads. Addition of functionally active anti-PD-L1 antibodies to CD4 cultures stimulated with □CD3 and PD-L1Fc coated beads increases CD4 proliferation (measured by either total CD4 number or percentage of the proliferation marker Ki67) (FIG. 7A) as compared to cultures with no antibody added. Addition of functionally active blocking anti-PD-L1 antibodies to CD4 cultures with □CD3 and PD-L1Fc coated beads also increases cytokine secretions by CD4 (measured by ELISA of accumulated IFN□ and IL-2 in the supernatant).

When PBMC isolated from whole blood are stimulated with the super antigen Staphylococcus Enterotoxin B (SEB) in the presence of anti-PD-L1 blocking antibodies, increase in cytokine secretions is observed. Supernatants of PBMC (previously frozen) cultured with SEB for 48 hours were collected, and IFN□ and IL-2 were measured by ELISA. No increase in T cells numbers were observed, but significant increases in the levels of IFN□ and IL-2 were observed in cultures of several anti-PD-L1 antibodies when compared to controls where no antibodies were added (FIG. 7B).

In addition, an increase in CD4 proliferation and activation is also observed in mixed-lymphocyte reaction (MLR) of CD4 T cells and mo-DC cultured in the presence of anti-PD-L1 blocking antibodies. Several of anti-PD-L1 antibodies increased CD4 proliferation in MLR when compared to cultures where no antibody was added (FIG. 7C). These antibodies also increased IFN□ and IL-2 secretion as evaluated by ELISA.

IL15 Increases Anti-PD-L1 Antibodies Effects on T Cell Proliferation and Activation in vitro

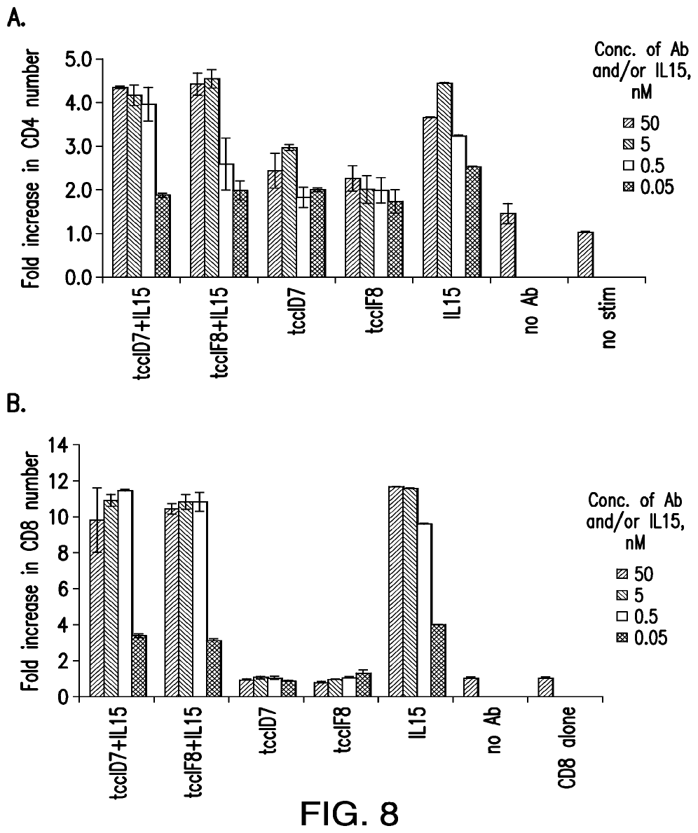

MLR of CD4 T cells and mo-DC in the presence of both anti-PD-L1 blocking antibodies and the cytokine IL15 resulted in significant increases in CD4 proliferation (FIG. 8A), IFN□ and IL-2 secretions when compared to cultures of CD4 and mo-DC with anti-PD-L1 antibodies alone. IL15 was added at equimolar concentrations as anti-PD-L1 antibodies in these assays. At lower anti-PD-L1 antibody and IL15 concentrations (0.5 nM, FIG. 8A), some synergistic effect on CD4 proliferation was observed.

Negatively purified CD8 from whole blood stimulated in vitro with aCD3 and PD-L1Fc coated beads also responds to IL15 in a dose-dependent fashion. Addition of IL15 to cultures of CD8 with aCD3 and PD-L1Fc coated beads and anti-PD-L1 antibodies resulted in large increases in CD8 proliferation (FIG. 8B).

### Anti-PD-L1-IL15 Fusion Protein Targets IL15 to PD-L1-Expressing Antigen Presenting Cells and Increases Proliferation and Activation of Responding CD8 Cells

Anti-PD-L1 antibody and IL15 fusion protein was constructed by linking the Fc domain of the antibody to the sushi-domain of IL15R and to IL15 molecule itself. The fusion of the IL15Rα sushi domain, IRD-11 exone3, linker and IL15 (designated “SD15”) is provided as SEQ ID NO:261. SD15 was appended to the heavy chain c-terminal of conventional IgG. The fusion protein with IL15Rα sushi domain, IRD-11 exone3, linker and IL15 was appended to the heavy chain c-terminal of tccλD7 variable domain and IgG1 C_{H}1-C_{H}2-C_{H}3 variable domain (SEQ ID NO:262). The construct also included a K to S replacement at the end of the IgG1 heavy chain (1) to diminish the possibility of “G-K” cleavage; (2) to add the cloning site (BamHI) to the vector.

The light chain is that of a conventional antibody. Both the light chain and fusion heavy chain with or without LALA mutant were inserted to Dyax pBh1 vector for expression.

This fusion molecule is designate anti-PD-L1-sushi domain-IL15 or anti-PD-L1-SD15. A different version of the fusion protein where IL15 was linked to the Fc instead of the sushi domain was also constructed, and as this fusion protein did not have IL15 functional activity we used this protein as negative control in some assays (termed anti-PD-L1-SD15neg).

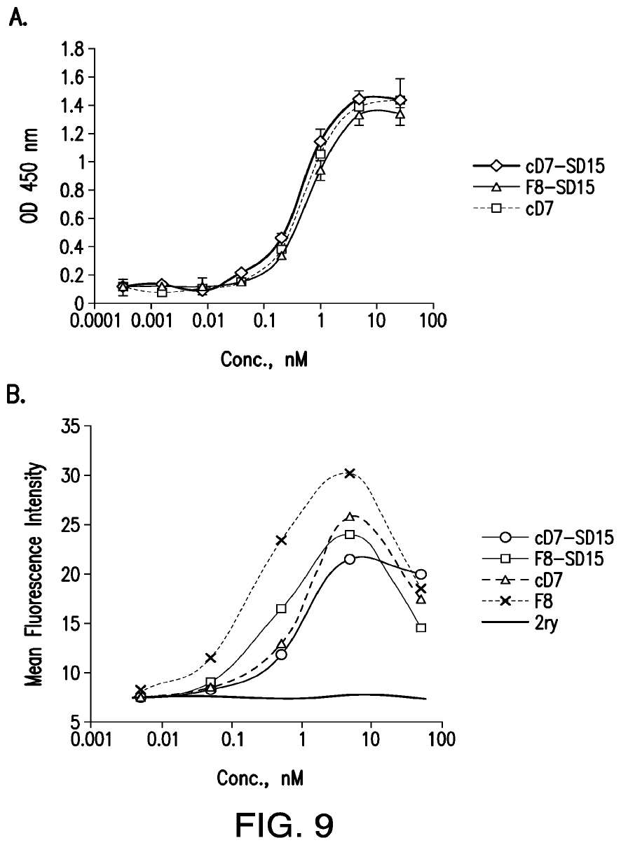

No significant change was observed when binding of anti-PD-L1-SD15 fusion proteins were compared to anti-PD-L1 antibodies in solid-phase PD-L1Fc binding ELISA assay (FIG. 9A). Some changes in binding affinity to activated CD4 cells expressing PD-L1 was observed when binding of anti-PD-L1-SD15 proteins were compared to their respective original anti-PD-L1 antibodies (FIG. 9B). Anti-PD-L1-SD15 proteins have lower affinity to cells expressing PD-L1 when compared to their respective anti-PD-L1 antibodies; although, there might be differences in binding of the secondary antibody to the bound anti-PD-L1-SD15 versus bound anti-PD-L1 on the surface of cells.

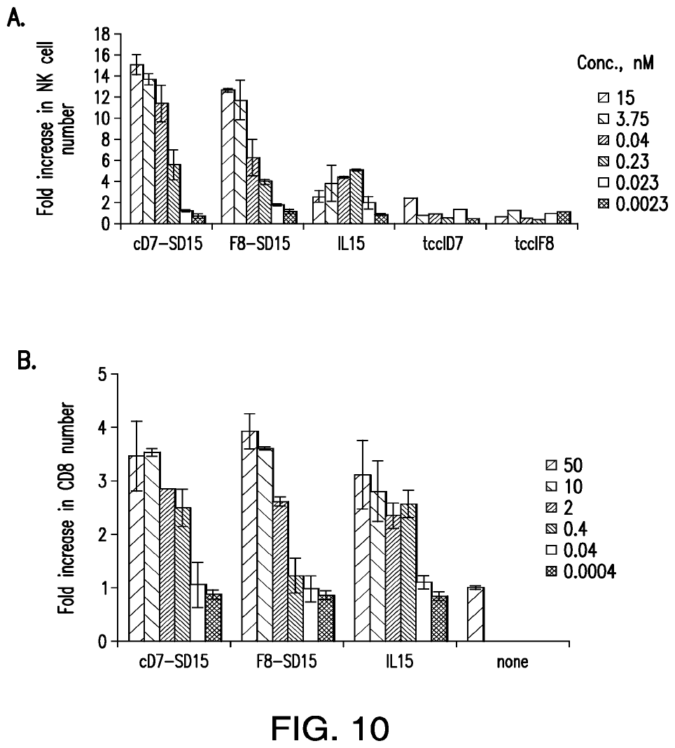

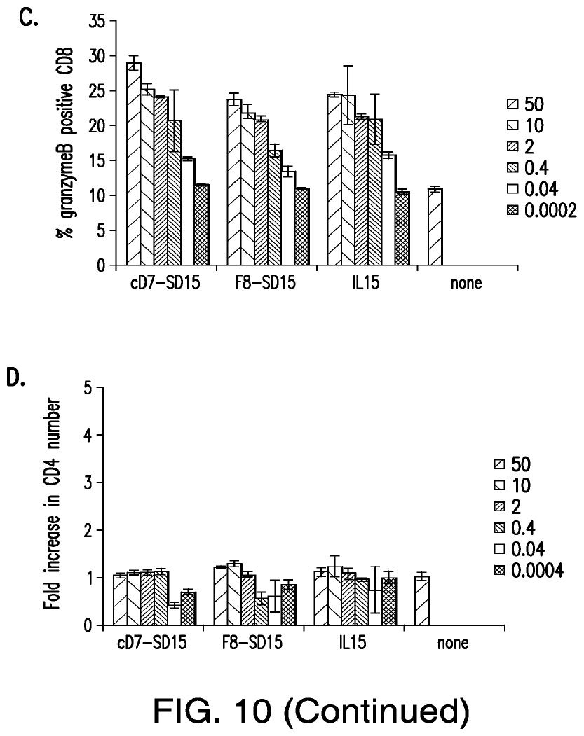

To evaluate the function of IL15 of anti-PD-L1-SD15 fusion proteins, PBMC isolated from whole blood was cultured in the presence of either anti-PD-L1-SD15 fusion proteins or IL15. No other stimulations were added to the cultures. Anti-PD-L1-SD15 fusion proteins increased NK cell number (FIG. 10A), increased CD8 proliferation (FIG. 10B) and activation (measured by % of granzymeB positive CD8, FIG. 10C) similarly as IL15. No significant increase in CD4 numbers were observed for all cultures (FIG. 10D).

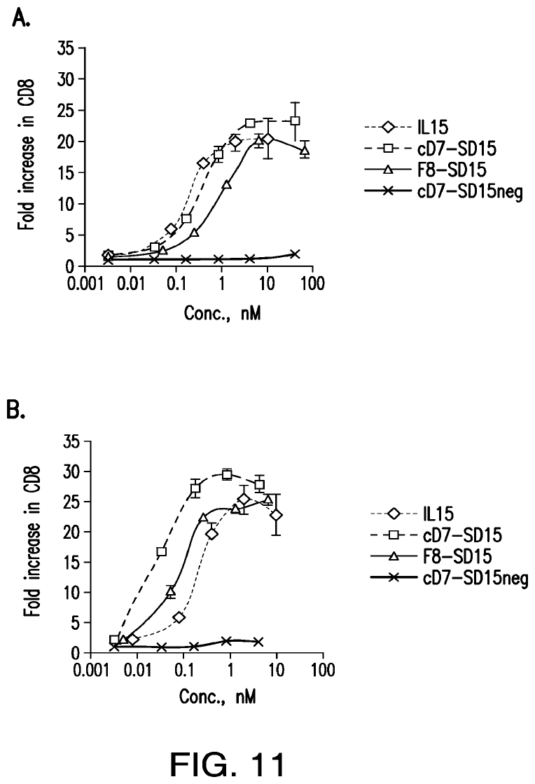

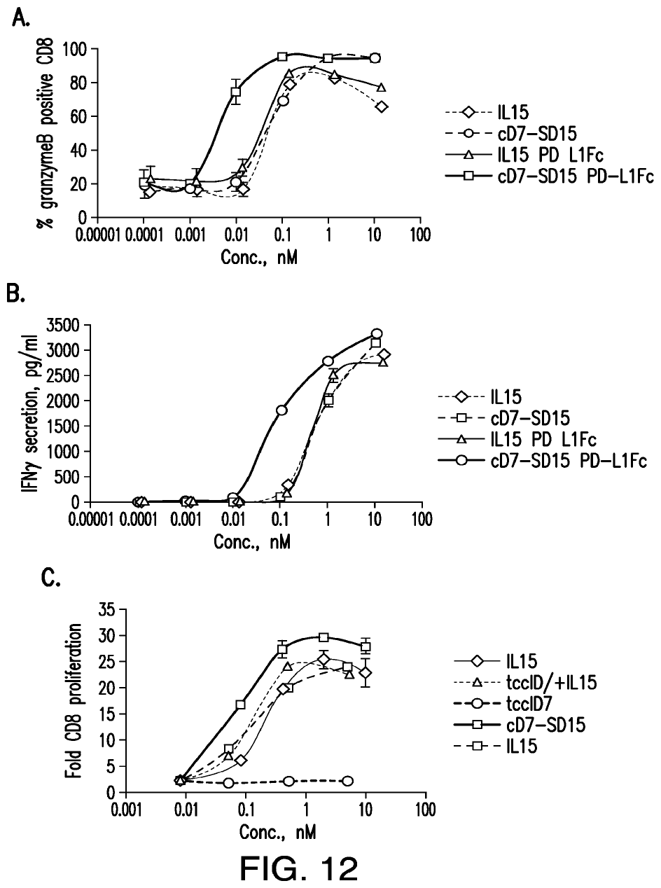

To assess anti-PD-L1-SD15 activity on CD8, these fusion proteins were added to CD8 cultures in the presence of either □CD3 or □CD3 and PDL1Fc coated beads. Anti-PD-L1-SD15 increased CD8 proliferation significantly when PDL1Fc was present on the antigen presenting cells, □CD3 and PDL1Fc coated beads in this case (FIG. 11A, no PD-L1Fc versus FIG. 11B, with PD-L1Fc on the beads). Moreover, significant increase of CD8 activation was also observed. cD7-SD15 lowers the effective dose needed to activate CD8 as measured by increase in % of granzymeB positive CD8 cells (FIG. 12A) and IFN□ secretion (FIG. 12B) by about ten-fold. cD7-SD15 also increases maximum level of CD8 activation when compared to IL15 (FIG. 12A and B). When compared to addition of anti-PD-L1 antibody plus free IL15, the anti-PD-L1-SD15 fusion protein increased CD8 proliferation to a level higher than the combination added separately (FIG. 12C). These properties of anti-PD-L1-SD15 fusion protein will be beneficial in the setting of immunotherapy as lower doses of anti-PD-L1-SD15 fusion protein can be used to achieve a higher level of CD8 activation and proliferation. The high amplified response of CD8 to anti-PD-L1-SD15 fusion protein in cases where the antigen presenting cells express PD-L1 will be advantageous in achieving selective CD8 activation.

### Cytotoxicity of Anti-PD-L1-IL15 Fusion Protein

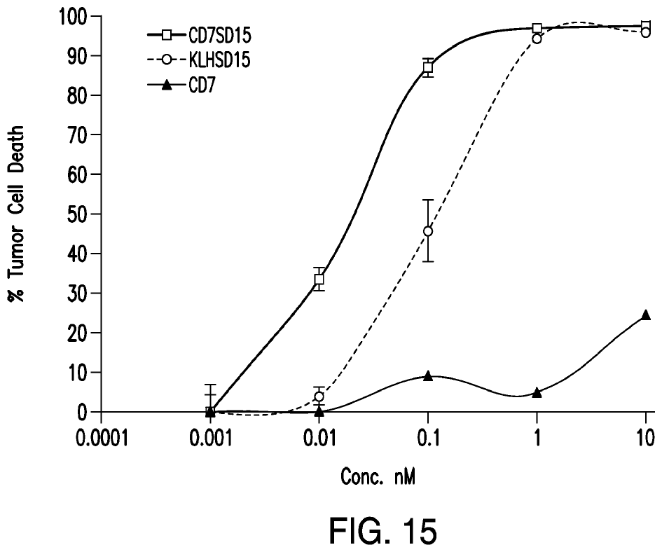

To determine whether anti-PD-L1-SD15 fusion protein will increase IL15 induced cytotoxicity of CD8 T cells to PD-L1 expressing tumor cells, CD8 T cells were co-cultured with human PD-L1 expressing MAD-MB-231 tumor cells in the presence of anti-PD-L1-SD15 fusion protein or anti-KLH-SD15, which has no binding activity to PD-L1 expressing tumor cells, for 7 days prior to the measurement of tumor cell death. Human CD8 T cells and the tumor cells were co-cultured in IMDM supplemented with 10% FBS for 7 days. Tumor cell killing activity was assessed by the measurement of the number of dead tumor cells stained by Viability Dye eFluor 780 in FACS. The CD8 T cell mediated cytotoxicity of MDA-MB-231 was significantly enhanced by anti-PD-L1-SD15 fusion protein in comparison to the treatment with anti-KLH-SD15 in the co-culture (FIG. 15). Moreover, PD-L1-SD15 fusion protein cD7-SD15 significantly increased the survival rate of mice bearing PD-L1 expressing tumor cells in the tumor model of mice intravenously injected with murine CT26 colon tumor cells in comparison to the mice treated with vehicle or PD-L1-SD15 fusion protein sD7-SD15, which does not have binding activity to murine PD-L1 (FIG. 16). These results indicate that the targeting IL15 stimulated immunological effector cells to PD-L1 overexpressed tumor sites by the bifunctional anti-PD-L1-SD15 fusion protein has advantage to enhance antitumor immunity while minimize side effects. This type of bifunctional antibody cytokine fusion proteins has potential as novel immunomodulatory therapeutics to achieve greater antitumor efficacy in the control of tumor progression.

### Affinity Maturation

Variants of the tccλD7 heavy chain were produced by introducing amino acid substitutions at three of the methionine positions in CDR-1H and screening for improved affinity. More particularly, a library containing about 1×10⁸ variants of CDRH1 of tccλD7 was generated in which the first, second and fourth methionine positions were simultaneously varied. The library was panned on recombinant human PDL1-Fc (PDL1 ECD and human Fc fusion protein, Q9NZQ7) or murine PDL1-Fc (Q9EP73) which were immobilized on immune-tubes for four rounds. The ELISA positive clones from rounds 3 and 4 were sequenced. The unique clones were compared by competition ELISA. Table 4 shows the amino acid substitutions observed in 25 variants obtained from the screen, with SEQ ID NOs: for the affinity matured CDR-1H sequences and heavy chain variable domains containing the CDRs. The amino acid sequences of these variants are also set forth the sequence listing as indicated in Table 1.

TABLE 4
CDR-H1 sequences of affinity matured variants of tccλD7.

| | | | | | | | | | | | SEQ ID NO | |
|---|---|---|---|---|---|---|---|---|---|---|---|---|
| tccλD7 | G | F | T | F | S | M | Y | M | M | M | CDR-1H | VH |
| #101 | — | — | — | — | — | A | — | A | — | A | 264 | 265 |
| #102 | — | — | — | — | — | A | — | R | — | F | 266 | 267 |
| #103 | — | — | — | — | — | A | — | L | — | V | 268 | 269 |
| #104 | — | — | — | — | — | A | — | V | — | F | 270 | 271 |
| #105 | — | — | — | — | — | A | — | V | — | S | 272 | 273 |
| #106 | — | — | — | — | — | G | — | L | — | V | 274 | 275 |
| #107 | — | — | — | — | — | G | — | Q | — | L | 276 | 277 |
| #108 | — | — | — | — | — | G | — | S | — | F | 278 | 279 |
| #109 | — | — | — | — | — | G | — | W | — | A | 280 | 281 |
| #110 | — | — | — | — | — | Q | — | L | — | Y | 282 | 283 |
| #111 | — | — | — | — | — | Q | — | V | — | F | 284 | 285 |
| #112 | — | — | — | — | — | Q | — | Y | — | Y | 286 | 287 |
| #113 | — | — | — | — | — | S | — | L | — | S | 288 | 289 |
| #114 | — | — | — | — | — | S | — | L | — | V | 290 | 291 |
| #115 | — | — | — | — | — | S | — | L | — | T | 292 | 293 |
| #116 | — | — | — | — | — | S | — | Q | — | V | 294 | 295 |
| #117 | — | — | — | — | — | S | — | S | — | A | 296 | 297 |
| #118 | — | — | — | — | — | S | — | V | — | F | 298 | 299 |
| #119 | — | — | — | — | — | S | — | V | — | S | 300 | 301 |
| #120 | — | — | — | — | — | S | — | V | — | Y | 302 | 301 |
| #121 | — | — | — | — | — | S | — | Y | — | F | 304 | 305 |
| #122 | — | — | — | — | — | S | — | Y | — | V | 306 | 307 |
| #123 | — | — | — | — | — | Y | — | S | — | V | 308 | 309 |
| #124 | — | — | — | — | — | W | — | L | — | A | 310 | 311 |
| #125 | — | — | — | — | — | W | — | Q | — | S | 312 | 313 |

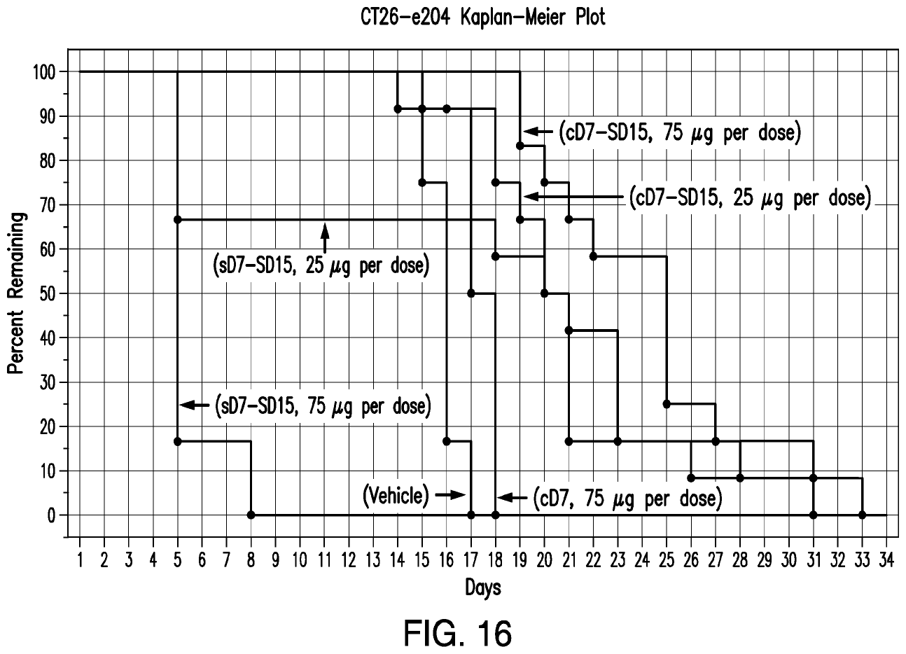

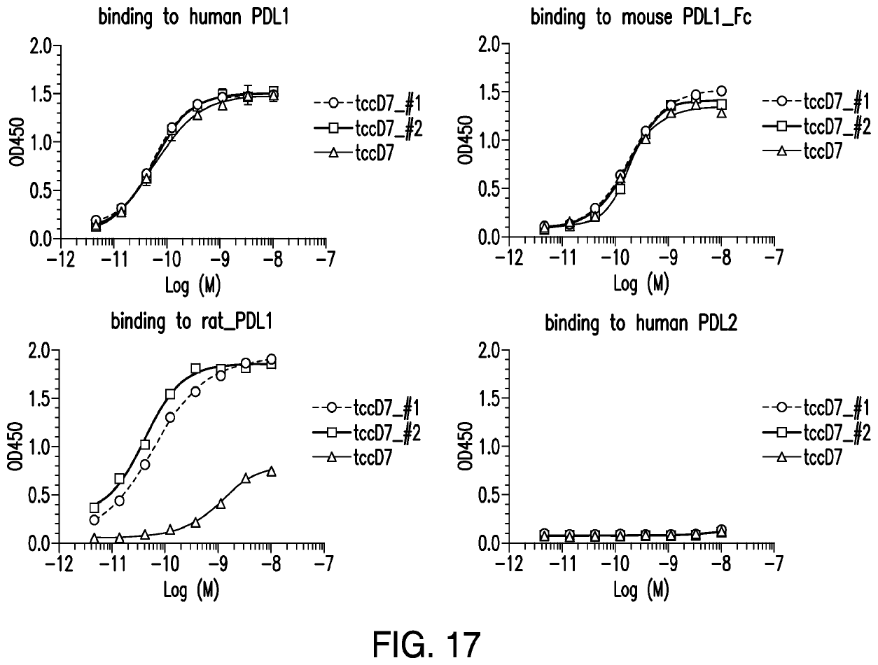

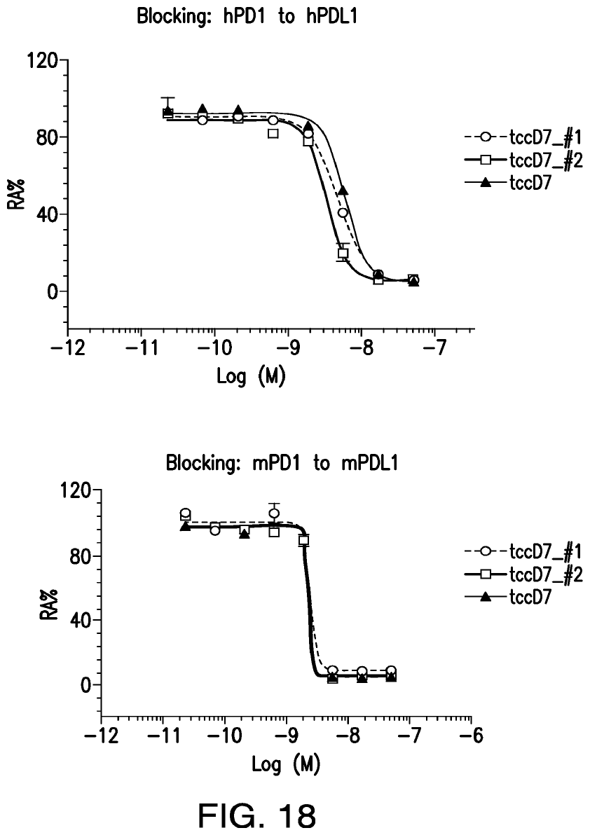

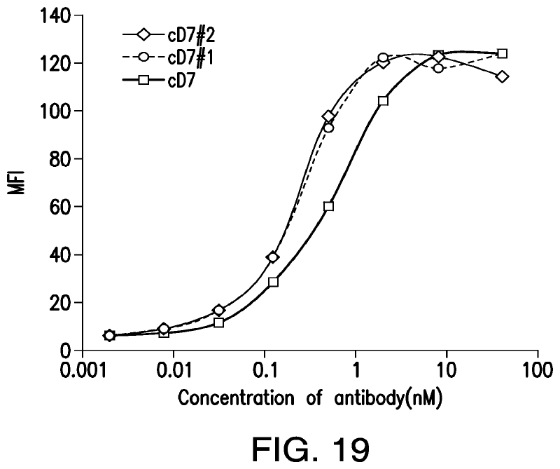

Two variants, tccD7_#114 and tccD7_#102 (also respectively referred to herein as tccD7_#1 and tccD7_#2) were converted to IgG and also to an IgG form containing two Leu-Ala substitutions in the hinge region for reduced ADCC, as described elsewhere herein. The antibodies were expressed and purified for the further characterization. Improved binding to soluble PDL1 is shown in FIG. 17 for the two affinity matured variants. FIG. 18 shows the two variants blocked binding of human PD1 to human PDL1 (left panel) and blocked binding of mouse PD1 to mouse PDL1 (right panel). The variants wlao demonstrated higher binding activity to MDA-MB-231 cells compared to the parent (FIG. 19).

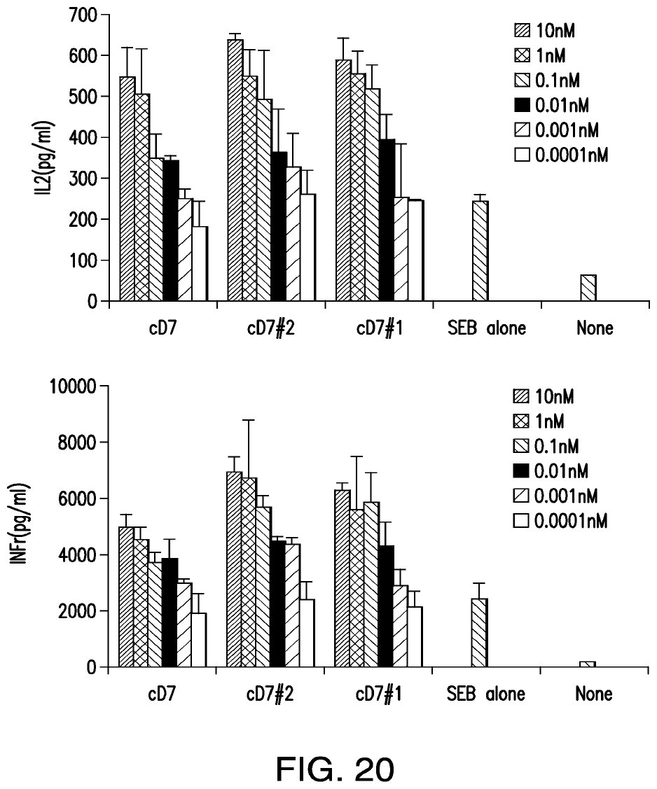

The affinity matured variants were tested for their ability to promote production of Th1 cytokines IL2 and IFNγ. PBMC isolated from whole blood were stimulated with the super antigen Staphylococcus Enterotoxin B (SEB, 0.1 ug/mL) in the presence of anti-PD-L1 antibodies. Supernatants of PBMC cultured with SEB for 7 days were collected, and IFNγ□ and IL-2 were measured by ELISA. Significant increases in the levels of IFNγ and IL-2 were observed in cultures with the variants of anti-PD-L1 antibodies cD7#1 and #2 when compared to cD7 (FIG. 20).

Several fusion protein variants comprising a PD-L1 binding domain, an IL15R a sushi domain and IL15 were constructed. Certain constructs include a linker between the IL15R a sushi domain and the IL15 portion. In one construct, 11 amino acids of exon 3 present in the c-terminal of the IL15 receptor α sushi domain were replaced with “GS” linkers of various lengths. GS linkers include SGGSGGGGSGGGSGGGGS (SEQ ID NO:324; 18 amino acids), SGGSGGGGSGGGSGGGGSLQ (SEQ ID NO:314; 20 amino acids), SGGGGSGGGGSGGGGSGGGGSGGGG (SEQ ID NO:316; 25 amino acids), SGGGGSGGGGSGGGGSGGGGSGGGGSGGGG (SEQ ID NO:318; 30 amino acids). SGGGGSGGGGSGGGGSGGGGSGGGGSGGGGSGGGGSGGGG (SEQ ID NO:320; 40 amino acids), and SGGGGSGGGGSGGGGSGGGGSGGGGSGGGGSGGGGSGGGGSGGGGSGGGG (SEQ ID NO:322; 50 amino acids) in constructs having SEQ ID NOS:325, 315, 317, 319, 321, and 323, respectively.

Fusion proteins were expressed in HEK293 cells, transiently or stably, and purified by protein A column chromatography according to manufacturers instructions. In certain experiments, to stabilize the association between the Ig heavy and light chain constant domains of the anti-PD-L1 portion of the molecule, the C-terminal serine of the lambda light chain was deleted, referred to herein by the designation “ds.”

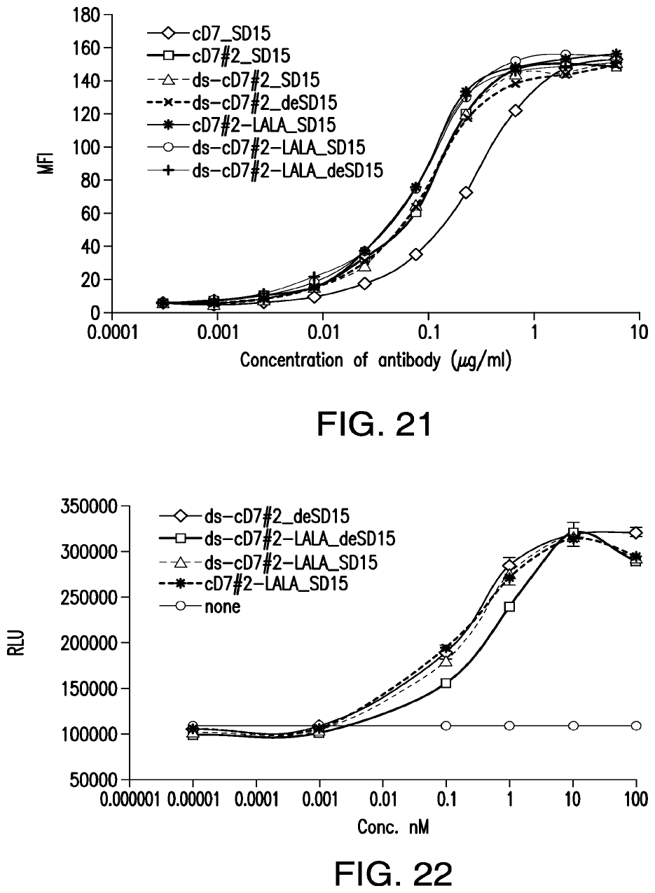

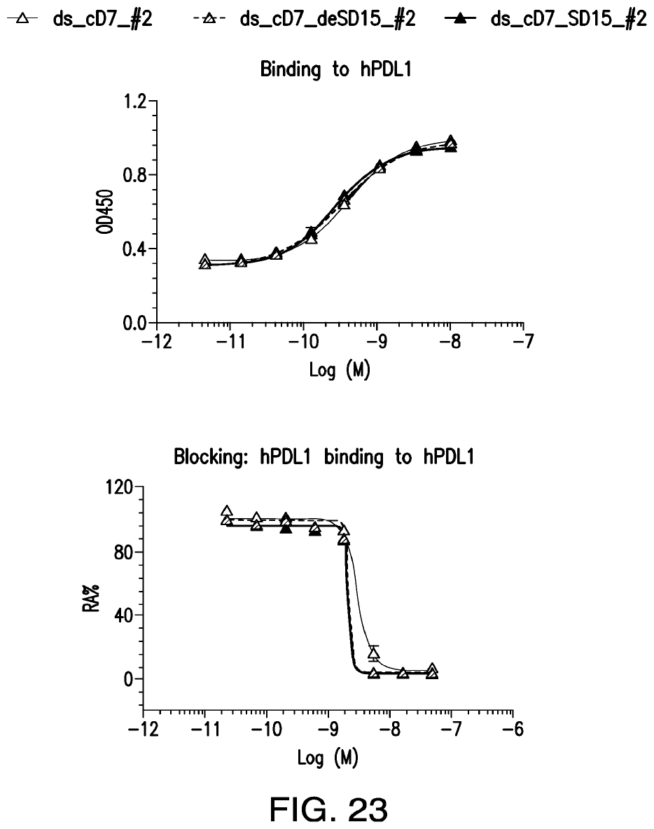

Fusion proteins containing the tccλD7 affinity matured variant #102 with the sushi domain and IL15 (SEQ ID NO:325) were tested for binding to MDA-MB-231 by flow cytometry. All demonstrated improved binding compared to the fusion protein containing tccλD7 (FIG. 21). The fusion proteins containing the tccλD7 affinity matured variant #102 were also confirmed to have stimulatory activity on IL15-responsive human megakaryoblastic leukemia cells. Cells were cultured with anti-PD-L1-SD15 fusion proteins in RPMI 1640 supplemented with 10% FBS and 20% conditioned medium of human bladder carcinoma 5637 cells for 48 hours. Cell proliferation was measured as Relative Luminescence Units (RLU) by CellTiter-Glo® Luminescent Cell Viability Assay (FIG. 22).

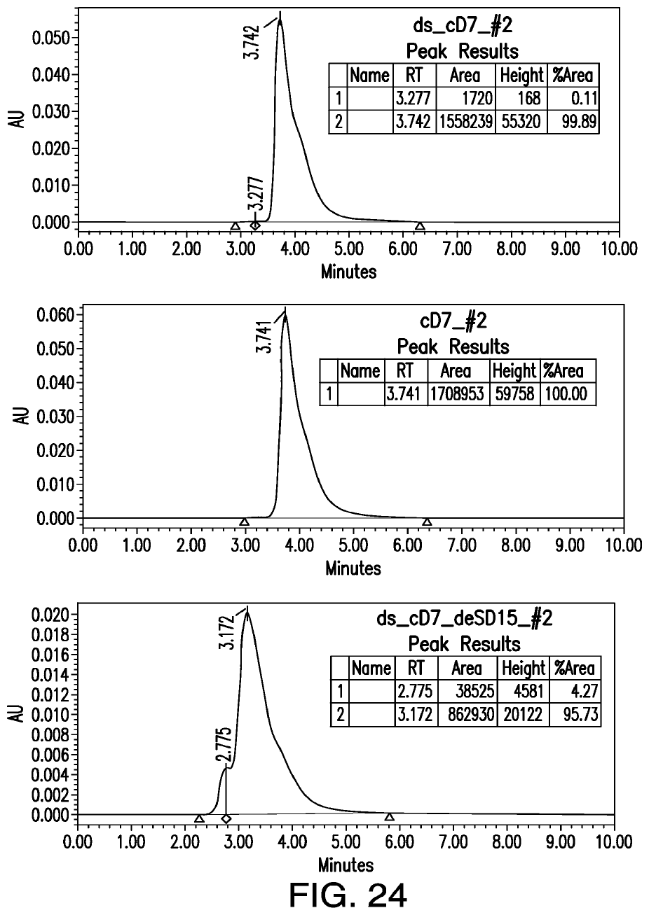

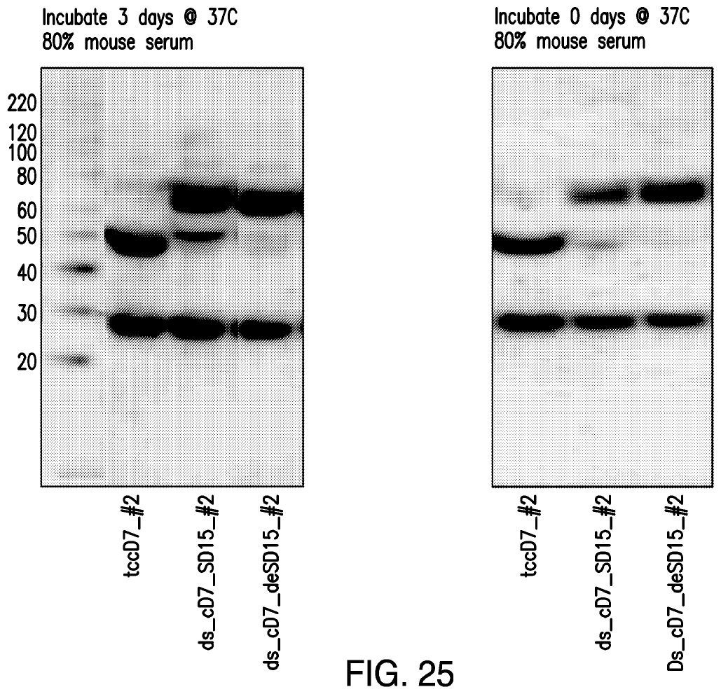

Analysis by size exclusion chromatography showed less than 5% aggregation (FIG. 24) and improved serum stability of the expressed fusion protein (FIG. 25).

1-34. (canceled)

35. A fusion protein comprising:
(i) an anti-PD-L1 antibody or fragment thereof;
(ii) an IL-15 receptor (IL-15R) alpha sushi domain comprising amino acids 1-61 of SEQ ID NO:261; and
(iii) IL-15 comprising amino acids 96-209 of SEQ ID NO:261, an amino acid sequence at least 95% identical to IL-15, or an IL-15R binding fragment thereof.

36. The fusion protein of claim 35, further comprising a flexible linker joining the IL-15R alpha sushi domain to the IL-15 or IL-15R-binding fragment thereof.

37. The fusion protein of claim 36, wherein the flexible linker comprises 15-20 amino acids, which are predominantly serine and glycine.

38. The fusion protein of claim 35, the fusion protein further comprising sequence SEQ ID NO: 261.
